(19) United States
(12) Patent Application Publication
(10) Pub. No.: US 2020/0057068 A1
(43) Pub. Date: Feb. 20, 2020

(54) Determining a Cancer Prognosis

(71) Applicant: The Council of the Queensland Institute of Medical Research, Herston, Queensland (AU)

(72) Inventors: Richard Lobb, Herston, Queensland (AU); Antoine Leimgruber, Vevey (CH); Andreas Möller, Herston, Queensland (AU)

(21) Appl. No.: 16/463,780

(22) PCT Filed: Nov. 24, 2017
(86) PCT No.: PCT/AU2017/051298

§ 371 Date: May 23, 2019

(30) Foreign Application Priority Data
Nov. 24, 2016 (AU) 2016904832

(51) Int. Cl. G01N 33/574

(52) U.S. Cl. CPC G01N 33/57423; G01N 2560/00

(57) ABSTRACT

Provided herein are methods of determining the aggressiveness, prognosis and response to therapy for cancer, such as non-small cell lung carcinoma (NSCLC), which includes determining an expression level of one or a plurality of differentially expressed protein markers in an exosome sample from a subject. A method and agent for treating cancer are also provided.

### FIELD

THIS INVENTION relates to cancer. More particularly, this invention relates to methods of determining the prognosis of cancers, in particular lung cancer.

### BACKGROUND

Lung cancer is a leading cause of cancer death and disease burden in many countries. By way of example, lung cancer in Australia accounts for 1 in every 14 to deaths in men and 1 in every 25 deaths in women from any cause. The stratification of patients into responding and non-responding categories is currently not possible for lung cancer.

Surgery is regarded as the optimal treatment for early stage lung cancer in people who are sufficiently fit for surgical resection. Nonetheless, clinical staging is imperfect as people treated by curative intent still have a significant chance of recurrence. For instance in stage I, II, or IIIA non-small cell lung cancer (NSCLC), about 40 to 50% of patients with stage IB, 55 to 70% of stage II, and a greater percentage of those with stage IIIA NSCLC eventually recur and die of their disease despite potentially curative surgery. In recent times, more active platinum-based combinations and a number of large clinical trials demonstrating effectiveness of adjuvant chemotherapy for resected NSCLC have led to the use of adjuvant chemotherapy to improve the outcome in patients with completely resected NSCLC.

Currently, the pathologic (TNM) staging is the most important prognostic factor determining the likelihood of relapse for lung cancer. Genomic biomarkers have been investigated for their potential prognostic value³⁻⁵ but at this time none are routinely used in the clinic unlike breast cancer where FDA-approved tests are increasingly being utilised in patients (e.g., Oncotype DX). Similarly, other biomarkers, including protein expression and proteomics, have been proposed for use in lung cancer but are yet to be routinely clinically applied.

Accordingly, there remains a pressing need for accurate prognostic biomarkers after treatment with curative intent, as a significant proportion of patients with NSCLC who undergo complete resection or chemoradiation as primary treatment for apparently curable lung cancer, eventually relapse and recur. Prognostic factors are required for guiding clinicians in determining which patients may be benefit from adjuvant chemotherapy, and who will suffer potential chemotherapy related adverse effects without any benefit.

In addition to the above, conventional validated prognostic biomarkers generally require the performance of invasive biopsies. However, in NSCLC patients co-morbidities and general health problems make 20% of patients unsuitable for such biopsies. Furthermore, biopsies themselves may cause injury and inflammation, contributing to the morbidity and mortality of NSCLC patients. Because of this, an improved method of assessing patient outcome from minimally-invasive sampling, such as blood tests, is required.

### SUMMARY

The present invention broadly relates to determining expression levels of one or more exosomal proteins as prognostic markers of cancer progression in a subject. In some aspects, the invention also broadly relates to the treatment of cancer using such exosomal proteins to inform treatment selection or decision making. In a particular form, the cancer is a lung cancer, such as non-small cell lung cancer.

In a first aspect, the invention provides a method of determining the aggressiveness of a cancer in a subject, said method including the step of determining an expression level of one or a plurality of markers in an exosome sample of the subject, wherein the markers comprise one or more of those proteins listed in Table 1 and/or Table 2 and an expression level of the one or plurality of markers indicates or correlates with a level of aggressiveness of the cancer.

In a second aspect, the invention provides a method of determining a prognosis for a cancer in a subject, said method including the step of determining an expression level of one or a plurality of markers in an exosome sample of the subject, wherein the markers comprise one or more of those proteins listed in Table 1 and/or Table 2 and an expression level of the one or plurality of markers indicates or correlates with a less or more favourable prognosis for said cancer.

In one embodiment of the method of the above aspects, a relatively decreased expression level of the one or plurality of markers indicates or correlates with a more favourable prognosis and/or a less aggressive cancer; and/or a relatively increased expression level of the one or plurality of markers indicates or correlates with a less favourable prognosis and/or a highly aggressive cancer.

Suitably, the method of first and second aspects further includes the step of diagnosing said subject as having: (i) a highly aggressive cancer or a less aggressive cancer; and/or (ii) a less favourable prognosis or a more favourable prognosis.

In one embodiment of the method of the aforementioned aspects, the cancer prognosis or aggressiveness is used, at least in part, to determine a likelihood of metastasis of the cancer in said subject. Suitably, a relatively decreased expression level of the one or plurality of markers indicates or correlates with a decreased likelihood of metastasis of said cancer; and/or a relatively increased expression level of the one or plurality of markers indicates or correlates with an increased likelihood of metastasis of said cancer.

In a third aspect, the invention provides a method of predicting the responsiveness of a cancer to an anti-cancer treatment in a subject, said method including the step of determining an expression level of one or a plurality of markers in an exosome sample of the subject, wherein the markers comprise one or more of those proteins listed in Table 1 and/or Table 2 and an altered or modulated expression level of the one or plurality of markers indicates or correlates with relatively increased or decreased responsiveness of the cancer to the anti-cancer treatment.

With respect to the invention of the first, second and third aspects, the method suitably includes the further step of treating the cancer in the subject.

In a fourth aspect, the invention provides a method of treating cancer in a subject, said method including the step of determining an expression level of one or a plurality of markers in an exosome sample of the subject, wherein the markers comprise one or more of those proteins listed in Table 1 and/or Table 2, and based on the determination made, initiating, continuing, modifying or discontinuing an anti-cancer treatment.

Suitably, for the method of the third and fourth aspects, the anti-cancer treatment comprises administration to the subject of a therapeutically effective amount of an anti-cancer agent that decreases the expression and/or an activity of the one or plurality of markers.

In one embodiment of the method of the third and fourth aspects, the anti-cancer treatment comprises administration to the subject of a therapeutically effective amount of an anti-cancer agent that prevents or inhibits metastasis of said cancer.

In reference to the method of the third and fourth aspects, the anti-cancer agent is suitably an antibody or a small molecule (e.g., a small organic or inorganic molecule antagonist).

Suitably, the method of the aforementioned aspects further includes the step of obtaining the exosome sample from the subject.

With respect to the method of the aforementioned aspects, the one or plurality of markers are suitably selected from the group consisting of Galectin-3-Binding Protein, Transitional endoplasmic reticulum ATPase, Neutral alpha-glucosidase AB, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-2, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof. In one particular embodiment, the one or plurality of markers are selected from the group consisting of Galectin-3-Binding Protein, Transitional endoplasmic reticulum ATPase, Tenascin C, Proteasome subunit alpha type-2, Thrombospondin-1 and any combination thereof.

Suitably, the method of the aforementioned aspects further includes the step of comparing the expression level of the one or plurality of markers in the exosome sample to a reference exosomal expression level of the respective one or plurality of markers.

In a fifth aspect, the invention provides a method for identifying or producing an agent for use in the treatment of cancer in a subject including the steps of:

(a) contacting a cell that expresses a marker listed in Table 1 and/or Table 2; with a candidate agent; and

(b) determining whether the candidate agent modulates the expression and/or an activity of the marker.

In certain embodiments, the candidate agent, at least partly, reduces, eliminates, suppresses or inhibits the expression and/or the activity of the marker.

Suitably, the cancer of the aforementioned aspects is or comprises a lung cancer. Preferably, the lung cancer includes squamous cell carcinoma, adenocarcinoma, large cell carcinoma, small cell carcinoma and mesothelioma. Even more preferably, the lung cancer is non-small cell lung carcinoma.

Suitably, the subject of the above aspects is a mammal, preferably a human.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

The indefinite articles ‘a’ and ‘an’ are used here to refer to or encompass singular or plural elements or features and should not be taken as meaning or defining “one” or a “single” element or feature. For example, “a” cell includes one cell, one or more cells and a plurality of cells.

### BRIEF DESCRIPTION OF THE FIGURES

FIG. 1. Exosomes are secreted by NSCLC cells. A Protein identification of exosomes demonstrates the presence of exosome markers, and the absence of non-exosomal calnexin. B Exosomes secreted by NSCLC have the expected size distribution. C Hypoxia increases the secretion of exosomes, but does not modify exosome size range. D Hypoxia significantly increases exosome secretion of NSCLC cells. CL: cell lysate; E: exosome lysate.

FIG. 2. Hypoxia modifies exosome content. Exosomes were harvested from conditioned media from cells cultured for 24 hours under normoxic (21% O₂) or hypoxic (2% O₂) conditions. A Scanning electron microscopy demonstrates classical exosome morphology. B Quantitative mass spectrometry revealed 55 proteins that are commonly upregulated under hypoxia, n=5, FDR 1%. C,D protein targets were validated with western blotting and ELISA.

FIG. 3. Proteins upregulated correlate to patient disease progression. A Exosomes isolated from NSCLC patients show the expected size range and morphology. B, C Hypoxic protein markers identified in vitro are upregulated in patients that relapse within the first 18 months. D ROC curve of combined protein signature (GANAB, VCP, and Galectin-3-Binding Protein) for identifying patients that relapse within 12 months. E Disease free survival of patients in relation to their exosome content. Patients that had at least 2 of the above markers highly expressed progressed rapidly, compared to patients that had only one or no markers expressed in their exosomes.

FIG. 4. Other upregulated proteins identified in hypoxic exosomes have prognostic value. TNC was upregulated under hypoxia and is more abundant in exosomes of NSCLC patients that progress rapidly.

FIG. 5. Individual ROC and survival curves of proteins used in patient signature.

FIG. 6. Hypoxia-induced changes to the protein composition of NSCLC cell-derived exosomes. a, The morphology of isolated exosomes was assessed using transmission electron microscopy. Images of normoxic and hypoxic SKMES1-derived exosomes (Size bar 200 nm) also indicate clear upregulation of exosome concentration. b, Nanoparticle analysis using TRPS of exosomes isolated from 4 different NSCLC cell lines demonstrating the majority of exosomes have a size range between 30 and 150 nm. c, Quantitative mass spectrometry identified 32 proteins to be commonly upregulated in H358 and SKMES1 exosomes (FDR<0.1%; n=5). d, e, Mass spectrometry results were confirmed using Western blot analysis of VCP (FLOT1 is used as a loading control), and ELISA for MAC2BP, TNC, PSMA2, and THBS1 in H358, SKMES1, H23, and H1975 NSCLC cell lines (●—H358, ▪—SKMES1, ▴—H23, ♦—H1975). *p<0.05, **p<0.01.

FIG. 7. Hypoxic exosome signature prognosticates disease progression in NSCLC patients. a, b, Exosomes can be isolated from NSCLC plasma based on morphology as shown by TEM (size bar 200 nm), and size distribution of 20-150 nm. c, TRPS demonstrates that there is no difference in exosome concentration in plasma from healthy controls or patients that progress within 18 months or patients without relapse at 18 months. d, Exosomes isolated from NSCLC patients show an enrichment of VCP in patients that progress with 18 months compared to patients that did not relapse and healthy controls (FLOT1 is used as a loading control). e, The hypoxic exosome signature is upregulated in exosome derived from patients that progress with 18 months. f, The number of hypoxic protein markers that exceed Youden's index threshold value demonstrates a clear separation between patients that progress within 18 months or patients without relapse at 18 months. g, Kaplan-Meier shows a clear separation of patient DFS based on the abundance of proteins from the hypoxic exosome signature (≥3 markers that exceed the Youden's index value). h, ROC curve demonstrates that the hypoxic exosome signature is a perfect prognostic marker of disease progression (<18 months) in NSCLC patients, while exosome concentration does not have prognostic value. i, Kaplan-Meier curve demonstrates the hypoxic exosome signature also correlates with overall survival in NSCLC patients.

FIG. 8. The hypoxic exosome signature is derived from lung cells that have undergone EMT. a, GSEA identified the hallmark epithelial-to-mesenchymal transition gene set was significantly associated with exosomes derived from hypoxic NSCLC cells. b, Immunofluorescence of normal lung epithelial (30KT) and transformed lung mesenchymal cells (30KT^{p53/KRAS/LKB1}) demonstrating oncogenically induced phenotypic transition to a mesenchymal phenotype. c, western blot in cell lysates demonstrates the loss of the epithelial marker E-cadherin and gain of the mesenchymal marker vimentin in 30KT^{p53/KRAS/LKB1} cells. d, western blot of VCP in exosomes derived from epithelial (30KT) and mesenchymal (30KT^{p53/KRAS/LKB1}) lung cells (CD9 is used as a loading control). e, ELISA of MAC2BP, TNC, PSMA2, and THBS1 in exosomes derived from epithelial (30KT) and mesenchymal (30KT^{p53/KRAS/LKB1}) lung cells. *p<0.05, **p<0.01 ***p<0.001. f, Immunohistochemistry of primary tumours demonstrates the loss of E-cadherin expression correlates to the patients that were stratified into the high signature group (≥3 markers that exceed the Youden's index value).

FIG. 9. Confirmation that the hypoxic exosome signature prognosticates disease relapse in NSCLC patients. a, b, ¹⁸F-FDG PET/CT images of 2 patients (confirmation cohort) that are tracked in c at indicated points. c, In support of the discovery cohort, exosome concentration in patients that relapse within 18 months compared to patients that relapse after 18 months was similar, in particular patient 44 and 53 are indicated. d, The number of hypoxic protein markers that exceed Youden's index threshold value demonstrates a clear separation between patients that progress within 18 months or patients without relapse at 18 months. e, Kaplan-Meier plot of DFS of NSCLC patients that have low abundance or high abundance of hypoxic exosome proteins indicates a clear separation in DFS. f, ROC curve analysis again shows a perfect classification of patients that will progress within 18 months. g, Kaplan-Meier plot confirms the signature is also a prognostic marker of overall survival in NSCLC patients.

FIG. 10. Hypoxia increases exosome secretion from NSCLC cells. a, Exosome isolated from NSCLC cell lines express canonical exosome markers HSP70, FLOT1, and CD63. The cell marker CANX is only found in cell lysates, not exosome lysates. b, Hypoxia increases exosome secretion from NSCLC cell lines. n=3±SEM, *p<0.05, **p<0.01 ***p<0.001.

FIG. 11. Discovery cohort demonstrates exosomal proteins are associated with disease progression in NSCLC patients. a-e, Individual Kaplan-Meier and ROC curves of each protein in the hypoxic exosome signature.

FIG. 12. Gene set enrichment analysis (GSEA) identified gene sets that were significantly elevated in exosomes derived from hypoxic NSCLC cells. A, Heatmap of proteins identified in the EMT gene set. b-e, GSEA using the total exosome protein expression dataset against hallmark gene sets reveals that hypoxic exosomes are enriched in proteins associated with glycolysis, MYC targets, E2F targets, and xenobiotic metabolism (FDR<0.05). NES—Normalised enrichment score.

FIG. 13. Reduced E-cadherin expression is correlated to the number of signature proteins that exceeds Youden's index threshold values. a, table of IHC scores in reference to the signature score. b, Low E-cadherin IHC scores are associated more prominently with patients that relapse within 18 months.

FIG. 14. Upregulated signature proteins in the confirmation cohort correlates with DFS. a, western blot of VCP demonstrates an upregulation of patients that progress within 18 months compared to patients that progress after 18 months (FLOT1 is used as a loading control). b, individual signature values of patient 44 and 53, show patient 53 who progresses within 18 months has significantly elevated baseline levels of the signature proteins compared to patient 44.

### DETAILED DESCRIPTION

The present invention is at least partly predicated on the surprising discovery that hypoxia-induced exosomal proteins identified in vitro are accurate prognostic biomarkers of cancer progression and aggressiveness in patients.

In one aspect, the invention provides a method of determining the aggressiveness of a cancer in a subject, said method including the step of determining an expression level of one or a plurality of markers in an exosome sample of the subject, wherein the markers comprise one or more of those proteins listed in Table 1 and/or Table 2 and an expression level of the one or plurality of markers indicates or correlates with a level of aggressiveness of the cancer.

In a related aspect, the invention provides a method of determining a prognosis for a cancer in a subject, said method including the step of determining an expression level of one or a plurality of markers in an exosome sample of the subject, wherein the markers comprise one or more of those proteins listed in Table 1 and/or Table 2 and an expression level of the one or plurality of markers indicates or correlates with a less or more favourable prognosis for said cancer.

With respect to the above aspects, the one or plurality of markers are suitably selected from the group consisting of Galectin-3-Binding Protein, Transitional endoplasmic reticulum ATPase, Neutral alpha-glucosidase AB, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-2, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof. In one particular embodiment, the one or plurality of markers are selected from the group consisting of Galectin-3-Binding Protein, Transitional endoplasmic reticulum ATPase, Tenascin C, Proteasome subunit alpha type-2, Thrombospondin-1 and any combination thereof.

As generally used herein, an expression level of one or more of: (a) the 55 marker proteins identified as upregulated in Table 1; and (b) the 32 marker proteins identified as upregulated in Table 2; may refer to the expression level of a nucleic acid encoding said protein (e.g., RNA, mRNA and cDNA), the protein itself or both, unless otherwise specified.

As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression.

By “aggressiveness” and “aggressive” is meant a property or propensity for a cancer to have a relatively poor prognosis due to one or more of a combination of features or factors including: at least partial resistance to therapies available for cancer treatment; invasiveness; metastatic potential; recurrence after treatment; and a low probability of patient survival, although without limitation thereto.

In particular embodiments, the proteins provided herein, such as those provided in Table 1 and Table 2, are prognostic for aggressive disease, and in particular a shorter time to pathological recurrence and/or a shorter patient survival time. In further embodiments, the proteins provided herein, such as those provided in Table 1 and Table 2, correlate with or indicate metastatic cancer, and more particularly, metastatic NSCLC. In this regard, it will be apparent that a number of the 32 proteins provided in Table 2 are also listed in Table 1, with the exception of, for example, LTBP3.

Cancers may include any aggressive or potentially aggressive cancers, tumours or other malignancies such as listed in the NCI Cancer Index at http://www.cancer.gov/cancertopics/alphalist, including all major cancer forms such as sarcomas, carcinomas, lymphomas, leukaemias and blastomas, although without limitation thereto. These may include breast cancer, lung cancer inclusive of lung adenocarcinoma and mesothelioma, cancers of the reproductive system inclusive of ovarian cancer, cervical cancer, uterine cancer and prostate cancer, cancers of the brain and nervous system, head and neck cancers, gastrointestinal cancers inclusive of colon cancer, colorectal cancer and gastric cancer, liver cancer, kidney cancer, skin cancers such as melanoma and skin carcinomas, blood cell cancers inclusive of lymphoid cancers and myelomonocytic cancers, cancers of the endocrine system such as pancreatic cancer and pituitary cancers, musculoskeletal cancers inclusive of bone and soft tissue cancers, although without limitation thereto.

In particular embodiments, the cancer includes breast cancer, lung cancer, ovarian cancer, cervical cancer, uterine cancer, prostate cancer, cancer of the brain and nervous system, head and neck cancer, colon cancer, colorectal cancer, gastric cancer, liver cancer, kidney cancer, bladder cancer, skin cancer, pancreatic cancer, pituitary cancer or adrenal cancer. More preferably, the cancer is or comprises lung cancer, such as NSCLC.

In particular embodiments, the cancer of the aspects disclosed herein is, or comprises, a lung cancer. To this end, it would be apparent that lung cancer may include any aggressive lung cancers and cancer subtypes known in the art, such as non-small cell carcinoma (i.e., squamous cell carcinoma, adenocarcinoma and large cell carcinoma), small cell carcinoma and mesothelioma. In one preferred embodiment, the lung cancer is or comprises non-small cell lung carcinoma (NSCLC).

The terms “prognosis” and “prognostic” are used herein to include making a prognosis, which can provide for predicting a clinical outcome (with or without medical treatment), selecting an appropriate course of treatment (or whether treatment would be effective) and/or monitoring a current treatment and potentially changing the treatment. This may be at least partly based on determining the gene and/or protein expression levels of the one or plurality of markers by the methods of the invention, which may be in combination with determining the expression levels of additional protein and/or other nucleic acid biomarkers. A prognosis may also include a prediction, forecast or anticipation of any lasting or permanent physical or psychological effects of cancer suffered by the subject after the cancer has been successfully treated or otherwise resolved. Furthermore, prognosis may include one or more of determining metastatic potential or occurrence, therapeutic responsiveness, implementing appropriate treatment regimes, determining the probability, likelihood or potential for cancer recurrence after therapy and prediction of development of resistance to established therapies (e.g., chemotherapy). It would be appreciated that a positive prognosis typically refers to a beneficial clinical outcome or outlook, such as long-term survival without recurrence of the subject's cancer, whereas a negative prognosis typically refers to a negative clinical outcome or outlook, such as cancer recurrence or progression.

In one embodiment of the method of the two aforementioned aspects, a relatively decreased expression level of the one or plurality of markers indicates or correlates with a more favourable prognosis and/or a less aggressive cancer; and/or a relatively increased expression level of the one or plurality of markers indicates or correlates with a less favourable prognosis and/or a highly aggressive cancer.

In one particular embodiment, the cancer prognosis or aggressiveness is used, at least in part, to determine a likelihood of metastasis of the cancer in said subject.

As used herein, “metastasis” or “metastatic”, refers to the migration or transfer of malignant tumour cells, or neoplasms, via the circulatory or lymphatic systems or via natural body cavities, typically from the primary focus of tumour, cancer or a neoplasia to a distant site in the body, and the subsequent development of one or more secondary tumours or colonies thereof in the one or more new locations. “Metastases” refers to the secondary tumours or colonies formed as a result of a metastasis and encompasses micro-metastases as well as regional, including lymph node, and distant metastases.

Suitably, a relatively decreased expression level of the one or plurality of markers indicates or correlates with a decreased likelihood of metastasis of said cancer; and/or a relatively increased expression level of the one or plurality of markers indicates or correlates with an increased likelihood of metastasis of said cancer.

In one embodiment, the cancer prognosis or aggressiveness is used, at least in part, to determine whether the subject would benefit from treatment of the cancer. By way of example, a patient with a favourable prognosis and/or a less aggressive cancer may be less likely to suffer from rapid local progression of the cancer and/or metastasis and can be spared from more aggressive monitoring and/or therapy.

In another embodiment, the cancer prognosis or aggressiveness is used, at least in part, to develop a treatment strategy for the subject.

In one embodiment, the cancer prognosis or aggressiveness is used, at least in part, to determine disease progression or recurrence in the subject.

In one embodiment, the cancer prognosis or aggressiveness is used, at least in part, to determine an estimated time of survival.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

As used herein a “gene” is a nucleic acid which is a structural, genetic unit of a genome that may include one or more amino acid-encoding nucleotide sequences and one or more non-coding nucleotide sequences inclusive of promoters and other 5′ untranslated sequences, introns, polyadenylation sequences and other 3′ untranslated sequences, although without limitation thereto. In most cellular organisms a gene is a nucleic acid that comprises double-stranded DNA.

The term “nucleic acid” as used herein designates single- or double-stranded DNA and RNA. DNA includes genomic DNA and cDNA. RNA includes mRNA, RNA, RNAi, siRNA, cRNA and autocatalytic RNA. Nucleic acids may also be DNA-RNA hybrids. A nucleic acid comprises a nucleotide sequence which typically includes nucleotides that comprise an A, G, C, T or U base. However, nucleotide sequences may include other bases such as inosine, methylycytosine, methylinosine, methyladenosine and/or thiouridine, although without limitation thereto.

Also included are, “variant” nucleic acids that include nucleic acids that comprise nucleotide sequences of naturally occurring (e.g., allelic) variants and orthologs (e.g., from a different species) of nucleic acids that respectively encode the one or plurality of markers provided herein. Preferably, nucleic acid variants share at least 70% or 75%, preferably at least 80% or 85% or more preferably at least 90%, 91%, 92%, 93%, 94%, 95%, 96%, 97%, 98% or 99% sequence identity with a nucleotide sequence disclosed herein.

Also included are nucleic acid fragments. A “fragment” is a segment, domain, portion or region of a nucleic acid, which respectively constitutes less than 100% of the nucleotide sequence. A non-limiting example is an amplification product or a primer or probe. In particular embodiments, a nucleic acid fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000 and 7500 contiguous nucleotides of said nucleic acid.

As used herein, a “polynucleotide” is a nucleic acid having eighty (80) or more contiguous nucleotides, while an “oligonucleotide” has less than eighty (80) contiguous nucleotides. A “probe” may be a single or double-stranded oligonucleotide or polynucleotide, suitably labelled for the purpose of detecting complementary sequences in Northern or Southern blotting, for example. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “template” nucleic acid is a nucleic acid subjected to nucleic acid amplification.

By “protein” is meant an amino acid polymer. The amino acids may be natural or non-natural amino acids, D- or L-amino acids as are well understood in the art. As would be appreciated by the skilled person, the term “protein” also includes within its scope phosphorylated forms of a protein (i.e., a phosphoprotein) and/or glycosylated forms of a protein (i.e. a glycoprotein). A “peptide” is a protein having no more than fifty (50) amino acids. A “polypeptide” is a protein having more than fifty (50) amino acids.

Also provided are protein “variants” such as naturally occurring variants (e.g. allelic variants) and orthologs or isoforms of the one or plurality of markers provided herein, such as those listed in Table 1 and Table 2. Preferably, protein variants share at least 70% or 75%, preferably at least 80% or 85% or more preferably at least 90%, 91%, 92%, 93%, 94%, 95%, 96%, 97%, 98% or 99% sequence identity with an amino acid sequence of the one or plurality of markers disclosed herein or known in the art. To this end, Tables 1 and 2 also include Accession Numbers referencing an example of a protein sequence of the recited protein marker, as are well understood in the art and are incorporated by reference herein.

Also provided are protein fragments, inclusive of peptide fragments that comprise less than 100% of an entire amino acid sequence. In particular embodiments, a protein fragment may comprise, for example, at least 10, 15, 20, 25, 30 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 1050, 1100, 1150 and 1200 contiguous amino acids of said protein.

It would be appreciated by the skilled person that exosomes are small (i.e., typically 30-150 nm), cell-derived membrane vesicles of endocytic origin. They may contain lipids, nucleic acid and proteins, and are released into the extracellular environment upon fusion with the plasma membrane. Generally, exosomes are characterized by the presence of marker proteins, including CD63, CD9, HSP70, Flotillin-1 and TSG101, as well as their morphology and size.

In accordance with the methods of the present invention, an exosome sample containing one or more exosomes may comprise or be obtained from most biological fluids including, without limitation, blood, serum, plasma, ascites, cyst fluid, pleural fluid, peritoneal fluid, cerebral spinal fluid, tears, urine, saliva, sputum, nipple aspirates, lymph fluid, fluid of the respiratory, intestinal, and genitourinary tracts, breast milk, intra-organ system fluid, or combinations thereof. To this end, an exosome sample may be isolated or purified from a biological fluid or sample, such as those provided above, so as to facilitate the removal of contaminating proteins, lipoproteins etc.

To this end, an exosome or exosome sample may be isolated by any means known in the art, such as, but not limited to, ultracentrifugation, size-exclusion chromatography, exosome precipitation (e.g., ExoQuick from System Biosciences), affinity-based capture of exosomes (e.g., affinity purification with antibodies to CD63, CD81, CD82, CD9, Alix, annexin, EpCAM, and Rab5) and any combination thereof.

As would be understood by the skilled person, the gene and/or protein expression level of the one or more proteins provided herein may be relatively (i) higher, increased or greater; or (ii) lower, decreased or reduced when compared to an expression level in a control or reference sample, or to a threshold expression level. In one embodiment, an expression level may be classified as higher increased or greater if it exceeds a mean and/or median expression level of a reference population. In one embodiment an expression level may be classified as lower, decreased or reduced if it is less than the mean and/or median expression level of the reference population. In this regard, a reference population may be a group of subjects who have the same cancer type, subgroup, stage and/or grade as said mammal for which the expression level is determined.

Terms such as “higher”, “increased” and “greater” as used herein refer to an elevated amount or level of a nucleic acid and/or protein, such as in an exosome sample, when compared to a control or reference level or amount. The expression level of the nucleic acid and/or protein of the one or plurality of markers may be relative or absolute. In some embodiments, the gene and/or protein expression of the one or plurality of markers is higher, increased or greater if its level of expression is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400% or at least about 500% above the level of gene and/or protein expression of the respective or corresponding protein in a control or reference level or amount.

The terms, “lower”, “reduced” and “decreased”, as used herein refer to a lower amount or level of a nucleic acid and/or protein, such as in an exosome sample, when compared to a control or reference level or amount. The expression level of the nucleic acid and/or protein of the one or plurality of markers provided herein may be relative or absolute. In some embodiments, the gene and/or protein expression of the one or plurality of markers is lower, reduced or decreased if its level of expression is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the level or amount of the gene and/or protein expression of the respective or corresponding protein in a control or reference level or amount.

The term “control sample” typically refers to a biological sample, such as an exosome sample, from a (healthy) non-diseased individual not having cancer. In one embodiment, the control sample may be from a subject known to be free of cancer or a sample that was obtained from the subject at an earlier timepoint. Alternatively, the control sample may be from a subject in remission from cancer. The control sample may be a pooled, average or an individual sample. An internal control is a marker from the same biological sample (e.g., exosome sample) being tested.

As used herein, a gene and/or protein expression level may be an absolute or relative amount thereof. Accordingly, in some embodiments, the gene and/or protein expression level of the one or plurality of markers provided herein is compared to a control level of expression, such as the level of gene and/or protein expression of one or a plurality of “housekeeping” genes and/or proteins in an exosome sample of the subject.

In further embodiments, the gene and/or protein expression level of the one or plurality of markers is compared to a threshold level of expression, such as a level of gene and/or protein expression in an exosome sample. A threshold level of expression is generally a quantified level of gene and/or protein expression of the one or plurality of markers of the invention. Typically, a gene and/or protein expression level of the one or plurality of markers in an exosome sample that exceeds or falls below the threshold level of expression is predictive of a particular disease state or outcome. The nature and numerical value (if any) of the threshold level of expression will typically vary based on the method chosen to determine the expression of the one or more genes, or products thereof, used in determining, for example, a prognosis and/or a response to anticancer therapy, in the subject.

A person of skill in the art would be capable of determining a threshold level of gene and/or protein expression in an exosome sample that may be used in determining, for example, a prognosis and/or a response to anticancer therapy, using any method of measuring gene or protein expression known in the art, such as those described herein. In one embodiment, the threshold level is a mean and/or median gene and/or protein expression level (median or absolute) of the one or plurality of markers in a reference population, that, for example, have the same cancer type, subgroup, stage and/or grade as said subject for which the expression level is determined. Additionally, the concept of a threshold level of expression should not be limited to a single value or result. In this regard, a threshold level of expression may encompass multiple threshold expression levels that could signify, for example, a high, medium, or low probability of, for example, metastasis of the subject's cancer.

In one embodiment, a lower gene and/or protein expression level of the one or plurality of markers provided herein indicates or correlates with relatively increased responsiveness of the cancer to the anti-cancer treatment. In alternative embodiments, a lower gene and/or protein expression level of the one or plurality of markers provided herein indicates or correlates with relatively decreased responsiveness of the cancer to the anti-cancer treatment.

The terms “determining”, “measuring”, “evaluating”, “assessing” and “assaying” are used interchangeably herein and may include any form of measurement known in the art, such as those described hereinafter.

Determining, assessing, evaluating, assaying or measuring corresponding nucleic acids of the one or plurality of markers provided herein, such as RNA, mRNA and cDNA, may be performed by any technique known in the art. These may be techniques that include nucleic acid sequence amplification, nucleic acid hybridization, nucleotide sequencing, mass spectroscopy and combinations of any these.

Nucleic acid amplification techniques typically include repeated cycles of annealing one or more primers to a “template” nucleotide sequence under appropriate conditions and using a polymerase to synthesize a nucleotide sequence complementary to the target, thereby “amplifying” the target nucleotide sequence. Nucleic acid amplification techniques are well known to the skilled addressee, and include but are not limited to polymerase chain reaction (PCR); strand displacement amplification (SDA); rolling circle replication (RCR); nucleic acid sequence-based amplification (NASBA), Q-I replicase amplification; helicase-dependent amplification (HAD); loop-mediated isothermal amplification (LAMP); nicking enzyme amplification reaction (NEAR) and recombinase polymerase amplification (RPA), although without limitation thereto. As generally used herein, an “amplification product” refers to a nucleic acid product generated by a nucleic acid amplification technique.

PCR includes quantitative and semi-quantitative PCR, real-time PCR, allele-specific PCR, methylation-specific PCR, asymmetric PCR, nested PCR, multiplex PCR, touch-down PCR, digital PCR and other variations and modifications to “basic” PCR amplification.

Nucleic acid amplification techniques may be performed using DNA or RNA extracted, isolated or otherwise obtained from a cell or tissue source. In other embodiments, nucleic acid amplification may be performed directly on appropriately treated cell or tissue samples.

Nucleic acid hybridization typically includes hybridizing a nucleotide sequence, typically in the form of a probe, to a target nucleotide sequence under appropriate conditions, whereby the hybridized probe-target nucleotide sequence is subsequently detected. Non-limiting examples include Northern blotting, slot-blotting, in situ hybridization and fluorescence resonance energy transfer (FRET) detection, although without limitation thereto. Nucleic acid hybridization may be performed using DNA or RNA extracted, isolated, amplified or otherwise obtained from a cell or tissue source or directly on appropriately treated cell or tissue samples.

It will also be appreciated that a combination of nucleic acid amplification and nucleic acid hybridization may be utilized.

Determining, assessing, evaluating, assaying or measuring protein levels of the one or plurality of exosomal proteins may be performed by any technique known in the art that is capable of detecting such proteins whether on the surface or internally expressed in an exosome, or proteins that are isolated, extracted or otherwise obtained from the exosome sample of the subject. These techniques include antibody-based detection that uses one or more antibodies which bind the protein, electrophoresis, isoelectric focussing, protein sequencing, chromatographic techniques and mass spectroscopy and combinations of these, although without limitation thereto. Antibody-based detection may include flow cytometry using fluorescently-labelled antibodies, ELISA, immunoblotting, immunoprecipitation, radioimmunoassay (RIA) and immuncytochemistry, although without limitation thereto.

It will be appreciated that determining the expression of the one or plurality of markers provided herein may include determining both the nucleic acid levels thereof, such as by nucleic acid amplification and/or nucleic acid hybridization, and the protein levels thereof. Accordingly, detection and/or measurement of expression of the one or plurality of markers from the exosome sample of the subject may be performed by any of those methods or combinations thereof described herein (e.g measuring mRNA levels or an amplified cDNA copy thereof and/or by measuring a protein product thereof), albeit without limitation thereto.

In light of the foregoing, it will further be appreciated that an expression level of the one or plurality of markers provided herein may be an absolute or relative amount of an expressed gene or gene product thereof, inclusive of nucleic acids such as RNA, mRNA and cDNA, and/or protein.

Suitably, the method of the aforementioned aspects further includes the step of diagnosing said subject as having: (i) a highly aggressive cancer or a less aggressive cancer; and/or (ii) a less favourable prognosis or a more favourable prognosis.

In a further aspect, the invention provides a method of predicting the responsiveness of a cancer to an anti-cancer treatment in a subject, said method including the step of determining an expression level of one or a plurality of markers in an exosome sample of the subject, wherein the markers comprise one or more of those proteins listed in Table 1 and/or Table 2 and an altered or modulated expression level of the one or plurality of markers indicates or correlates with relatively increased or decreased responsiveness of the cancer to the anti-cancer treatment.

As would be understood by the skilled person, the expression level of a gene or protein may be deemed to be “altered” or “modulated” when the expression level is higher/increased or lower/decreased when compared to a control or reference sample or expression level, such as a threshold level. In one embodiment, the expression level may be classified as high if it is greater than a mean and/or median relative expression level of a reference population and the expression level may be classified as low if it is less than the mean and/or median expression level of the reference population. In this regard, a reference population may be a group of subjects who have the same cancer type, subgroup, stage and/or grade as said mammal for which the expression level is determined. Furthermore, the expression level may be relative or absolute.

Suitably, the one or plurality of markers are selected from the group consisting of Galectin-3-Binding Protein, Transitional endoplasmic reticulum ATPase, Neutral alpha-glucosidase AB, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-2, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof. In one particular embodiment, the one or plurality of markers are selected from the group consisting of Galectin-3-Binding Protein, Transitional endoplasmic reticulum ATPase, Tenascin C, Proteasome subunit alpha type-2, Thrombospondin-1 and any combination thereof.

In one embodiment, a higher expression level of the one or plurality of markers indicates or correlates with relatively increased responsiveness of the cancer to the anti-cancer treatment. In alternative embodiments, a higher expression level of the one or plurality of markers indicates or correlates with relatively decreased responsiveness of the cancer to the anti-cancer treatment.

With respect to the invention of the aforementioned aspects, the method suitably includes the further step of treating the cancer in the subject.

Further aspects of the invention relate to treatment of cancer in a subject.

In one particular aspect, the cancer treatment is performed in conjunction with determining an expression level of one or a plurality of markers in an exosome sample of the subject, wherein the markers comprise one or more of those proteins listed in Table 1 and/or Table 2, and based on the determination made, initiating, continuing, modifying or discontinuing the cancer treatment.

Suitably, the one or plurality of markers are selected from the group consisting of Galectin-3-Binding Protein, Transitional endoplasmic reticulum ATPase, Neutral alpha-glucosidase AB, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-2, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3, and any combination thereof. In one particular embodiment, the one or plurality of markers are selected from the group consisting of Galectin-3-Binding Protein, Transitional endoplasmic reticulum ATPase, Tenascin C, Proteasome subunit alpha type-2, Thrombospondin-1 and any combination thereof.

In this regard, it would be appreciated that those methods described herein for predicting the responsiveness of a cancer to an anti-cancer agent may further include the step of administering to the mammal a therapeutically effective amount of the anti-cancer treatment, such as an anticancer agent. In a preferred embodiment, the anticancer treatment is administered when the gene and/or protein expression level of the one or plurality of markers described herein indicates or correlates with relatively increased responsiveness of the cancer to the anti-cancer agent.

Suitably, the agent(s) is/are administered to a subject as a pharmaceutical composition comprising a pharmaceutically-acceptable carrier, diluent or excipient. In this regard, any dosage form and route of administration, such as those provided therein, may be employed for providing a subject with the composition of the invention.

Cancer treatments may include drug therapy, such as small organic or inorganic molecules, chemotherapy, antibody, nucleic acid and other biomolecular therapies, radiation therapy, surgery, nutritional therapy, relaxation or meditational therapy and other natural or holistic therapies, although without limitation thereto. Generally, drugs (e.g., small organic or inorganic molecules), biomolecules (e.g antibodies, inhibitory nucleic acids such as siRNA) or chemotherapeutic agents are referred to herein as “anti-cancer therapeutic agents” or “anti-cancer agents”.

Methods of treating cancer may be prophylactic, preventative or therapeutic and suitable for treatment of cancer in mammals, particularly humans. As used herein, “treating”, “treat” or “treatment” refers to a therapeutic intervention, course of action or protocol that at least ameliorates a symptom of cancer after the cancer and/or its symptoms have at least started to develop. As used herein, “preventing”, “prevent” or “prevention” refers to therapeutic intervention, course of action or protocol initiated prior to the onset of cancer and/or a symptom of cancer so as to prevent, inhibit or delay or development or progression of the cancer or the symptom.

The term “therapeutically effective amount” describes a quantity of a specified agent sufficient to achieve a desired effect in a subject being treated with that agent. For example, this can be the amount of a chemotherapeutic agent necessary to reduce, alleviate and/or prevent a cancer or cancer associated disease, disorder or condition. In some embodiments, a “therapeutically effective amount” is sufficient to reduce or eliminate a symptom of a cancer. In other embodiments, a “therapeutically effective amount” is an amount sufficient to achieve a desired biological effect, for example an amount that is effective to decrease or prevent cancer growth and/or metastasis.

Ideally, a therapeutically effective amount of an agent is an amount sufficient to induce the desired result without causing a substantial cytotoxic effect in the subject. The effective amount of an agent useful for reducing, alleviating and/or preventing a cancer will be dependent on the subject being treated, the type and severity of any associated disease, disorder and/or condition (e.g., the number and location of any associated metastases), and the manner of administration of the therapeutic composition.

Suitably, the anti-cancer therapeutic agent is administered to a mammal as a pharmaceutical composition comprising a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, liposomes and other lipid-based carriers, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunotherapeutic compositions, proteinaceous vaccines and nucleic acid vaccines.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention suitable for oral or parenteral administration may be presented as discrete units such as capsules, sachets or tablets each containing a pre-determined amount of one or more therapeutic agents of the invention, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association one or more agents as described above with the carrier which constitutes one or more necessary ingredients. In general, the compositions are prepared by uniformly and intimately admixing the agents of the invention with liquid carriers or finely divided solid carriers or both, and then, if necessary, shaping the product into the desired presentation.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

In particular embodiments, the anti-cancer treatment and/or agent may be directed at inhibiting the action of and/or decreasing the expression of the one or plurality of markers.

In other embodiments, the anti-cancer treatment and/or agent may be directed at preventing or inhibiting metastasis of the cancer.

In alternative embodiments, the anti-cancer treatment and/or agent may be directed at genes or gene products other than the one or plurality of markers of the invention. By way of example, the anti-cancer treatment may target genes or gene products that are known to interact, directly or indirectly, with the one or plurality of markers.

In a particular embodiment, the invention provides a “companion diagnostic” with respect to the cancer treatment, whereby the expression level of the one or plurality of markers of the invention provides information to a clinician or the like that is used for the safe and/or effective administration of said cancer treatment.

Suitably, the cancer is of a type hereinbefore described, albeit without limitation thereto.

Referring to the aforementioned aspects, the method suitably includes the initial step of obtaining the exosome sample from the subject, such as from those biological samples and/or isolation methods hereinbefore described.

In a further aspect, the invention provides a method for identifying or producing an agent for use in the treatment of cancer in a subject including the steps of:

(a) contacting a cell that expresses a marker listed in Table 1 and/or Table 2; with a candidate agent; and

(b) determining whether the candidate agent modulates the expression and/or an activity of the marker.

In certain embodiments, the candidate agent, at least partly, reduces, eliminates, suppresses or inhibits the expression and/or the activity of the marker.

Suitably, the agent possesses or displays little or no significant off-target and/or nonspecific effects.

Preferably, the agent is an antibody or a small molecule.

Suitably, the marker is selected from the group consisting of Galectin-3-Binding Protein, Transitional endoplasmic reticulum ATPase, Neutral alpha-glucosidase AB, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-2, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof. In one particular embodiment, the one or plurality of markers are selected from the group consisting of Galectin-3-Binding Protein, Transitional endoplasmic reticulum ATPase, Tenascin C, Proteasome subunit alpha type-2, Thrombospondin-1 and any combination thereof.

In embodiments relating to antibody inhibitors, the antibody may be polyclonal or monoclonal, native or recombinant. Well-known protocols applicable to antibody production, purification and use may be found, for example, in Chapter 2 of Coligan et al., CURRENT PROTOCOLS IN IMMUNOLOGY (John Wiley & Sons NY, 1991-1994) and Harlow, E. & Lane, D. Antibodies: A Laboratory Manual, Cold Spring Harbor, Cold Spring Harbor Laboratory, 1988, which are both herein incorporated by reference.

Generally, antibodies of the invention bind to or conjugate with an isolated protein, fragment, variant, or derivative of the marker. For example, the antibodies may be polyclonal antibodies. Such antibodies may be prepared for example by injecting an isolated protein, fragment, variant or derivative of the marker protein product into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, supra, and in Harlow & Lane, 1988, supra.

Monoclonal antibodies may be produced using the standard method as for example, described in an article by Kohler & Milstein, 1975, Nature 256, 495, which is herein incorporated by reference, or by more recent modifications thereof as for example, described in Coligan et al., CURRENT PROTOCOLS IN IMMUNOLOGY, supra by immortalizing spleen or other antibody producing cells derived from a production species which has been inoculated with one or more of the isolated marker protein products and/or fragments, variants and/or derivatives thereof.

Typically, the inhibitory activity of candidate inhibitor antibodies may be assessed by in vitro and/or in vivo assays that detect or measure the expression levels and/or activity of the marker protein in the presence of the antibody.

In some embodiments, modulators such as inhibitors may be rationally designed. These methods may include structural analysis of the marker and the design and/or construction of molecules that bind, interact with or otherwise modulate the activity of the marker. These methods may particularly include computer-aided three-dimensional modelling of the interaction between the candidate modulator and the marker.

In other embodiments, modulators such as small organic molecule inhibitors, this may involve screening of large compound libraries, numbering hundreds of thousands to millions of candidate inhibitors (chemical compounds including synthetic, small organic molecules or natural products, such as inhibitory peptides or proteins) which may be screened or tested for biological activity at any one of hundreds of molecular targets in order to find potential new drugs, or lead compounds. Screening methods may include, but are not limited to, computer-based to (“in silico”) screening and high throughput screening based on in vitro assays.

Typically, the active compounds, or “hits”, from this initial screening process are then tested sequentially through a series of other in vitro and/or in vivo tests to further characterize the active compounds. A progressively smaller number of the “successful” compounds at each stage are selected for subsequent testing, eventually leading to one or more drug candidates being selected to proceed to being tested in human clinical trials.

At the clinical level, screening a candidate agent may include obtaining samples from test subjects before and after the subjects have been exposed to a test compound. The levels in the samples, such as exosome samples, of marker protein may then be measured and analysed to determine whether the levels and/or activity of the marker protein changes after exposure to a candidate agent. By way of example, protein product levels in the samples may be determined by mass spectrometry, western blot, ELISA, electrochemistry and/or by any other appropriate means known to one of skill in the art.

In this regard, candidate agents that are identified of being capable of reducing, eliminating, suppressing or inhibiting the expression level and/or activity of the marker may then be administered to patients who are suffering from cancer. For example, the administration of a candidate agent which inhibits or decreases the activity and/or expression of the marker may treat the cancer and/or decrease the risk of cancer, if the increased activity of the biomarker is responsible, at least in part, for the progression and/or onset of the cancer.

With respect to the aforementioned aspects, the term “subject” includes but is not limited to mammals inclusive of humans, performance animals (such as horses, camels, greyhounds), livestock (such as cows, sheep, horses) and companion animals (such as cats and dogs). Preferably, the subject is a human.

All computer programs, algorithms, patent and scientific literature referred to herein is incorporated herein by reference.

For the present invention, the database accession number or unique identifier provided herein for a gene or protein, such as those presented in Table 1 and Table 2, as well as the gene and/or protein sequence or sequences associated therewith, are incorporated by reference herein.

So that preferred embodiments of the invention may be fully understood and put into practical effect, reference is made to the following non-limiting examples.

### Example 1

Recent data suggests that tumour hypoxia is a strong driving force for the secretion of factors that promote the metastatic dissemination^{8,9}. A critical component of secreted factors that are thought to be involved in enhancing metastasis is the release of exosomes. Increasing evidence suggests that the rich array of proteomic and genomic information carried by tumour-derived exosomes is a novel mechanism by which cancer cells modify surrounding stroma and malignant cell behaviour¹⁰. Exosomes can affect signalling processes involved in neo-angiogenesis¹¹, immune suppression¹², and induce drug resistance and oncogenic transfer¹³⁻¹⁵ Moreover, the ability of exosomes to induce systemic changes is thought to promote metastatic dissemination, which accounts for a majority of patient deaths¹⁶.

The transfer of oncogenic proteins by exosomes has also been reported¹⁴. Exosome transfer in glioma cells has recently been demonstrated to enhance tumorigenesis through delivery of a mutant epidermal growth factor receptor (EGFRvIII) isoform, resulting in increased expression of anti-apoptotic genes and enhanced proliferation¹⁴. Similarly, colon cancer cells with a mutant form of KRAS are capable of enhancing the three-dimensional growth of wild-type KRAS colon cells via exosomal transfer of mutant KRAS to the wild-type cells. Additionally, non-metastatic melanoma cells can be induced to become more metastatic by the uptake of exosomes derived from a highly metastatic melanoma cell line¹⁷. However, whether this change in metastatic potential is permanent remains unclear.

The protein and RNA content of exosomes typically varies significantly depending on the cell type, tissue, and microenvironment they originate from. For this reason, cancer-secreted exosomes and their molecular contents represent potential sources of biomarkers and therapeutic targets in cancer. Accordingly, the overall aim of this Example was to establish a means to non-invasively predict disease progression in NSCLC patients from their blood using exosomes.

Currently, there is a large unmet need to develop non-invasive and informative diagnostic markers for a variety of solid malignancies. The proteomic and RNA information contained in tumour-derived exosomes has generated significant interest for the use of exosomes as a non-invasive diagnostic tool. As exosome isolation techniques are now well established, and because exosomes are stable in bodily fluids, including serum, urine and saliva, they demonstrate great potential as reliable biomarkers of disease progression²³. Given that exosomes may provide molecular signatures of their cell of origin, proteomic and RNA analysis may also provide an efficient means to determine oncogenic mutations. Recently, it was shown that exosome-based proteins, in this case the presence of Glypican-1, can predict short disease-free survival in pancreatic cancer patients²⁴.

Moreover, exosomes derived from patients may prove useful in understanding the progression and treatment options for the disease. This has already been demonstrated with exosomes isolated from melanoma patients, which exhibited high protein content and elevated expression of TYRP2, VLA 4, and HSP70; proteins that were enriched in patients with a poor prognosis¹⁶. Furthermore, a number of different group have identified retrotransposon RNA transcripts, single-stranded DNA (ssDNA), mitochondrial DNA, and oncogene amplifications (i.e., cMyc) in microvesicles as well as double-stranded DNA (dsDNA) in exosomes²⁵. Amongst the oncogenes in exosomes, cMet (melanoma)¹⁶, mutated KRAS and p53 in pancreatic cancer²⁶ have so far been reported. Thus, given the presence of these specific exosomal biomolecules coupled to their known release by tumour cells, exosomes may prove a clinically useful enriched template for simplex or multiplexed diagnostic biomarkers²⁷, reviewed by²⁸.

### Materials and Methods

Cell Lines and Cell Culture

Human non-small cell lung cancer (NSCLC) cell lines were purchased from American Type Culture Collection (ATCC). All cell lines were confirmed by short tandem repeat (STR) profiling and were found to be negative for mycoplasma. All cells were maintained in a humidified incubator with 5% CO₂ at 37° C. SKMES1 cells were cultured in DMEM, supplemented with 10% FBS (Gibco, Thermo Fisher Scientific), and penicillin-streptomycin. All other cells were cultured in RPMI, supplemented with 10% FBS and penicillin-streptomycin. For hypoxia experiments, cells were cultured in a humidified incubator with 2% O₂ and 5% CO₂ at 37° C.

Exosome Isolation

Serum media was removed by washing cells twice with PBS and replacing with 15 mL of serum-free media. Media was conditioned for 24 hours at normoxia (21% 02), or hypoxia (21% 02). Conditioned media was aliquoted into falcon tubes and floating cells and debris was removed by centrifugation at 300×g at 4° C. for 10 minutes. The resulting supernatant was filtered through 0.22 μm filters to remove the remaining large particles. Clarified conditioned media was concentrated to 300-500 μL using a Centricon Plus-70 Centrifugal Filter (Ultracel-PL Membrane, 100 kDa) device at 3,500 g at 4° C. Exosomes were then purified using an OptiPrep density gradient. Concentrated media was overlaid on a discontinuous iodixanol gradient and centrifuged 16 hours at 100,000 g_{avg} (k-factor: 277.5) at 4° C. Exosome containing fractions were identified with tunable resistive pulse sensing (TRPS) and diluted to 20 mL in PBS and centrifuged at 100,000 g_{avg} for 2 hours at 4° C. The resulting pellet was resuspended in PBS for further analysis.

Electron Microscopy

Exosomes were visualized using transmission electron microscopy (TEM). Three μL of exosome suspension was fixed in 50-100 μL of 2% paraformaldehyde. A Two microliter aliquot was then transferred onto each of 2 Formvar-carbon coated electron microscopygrids and then covered for 20 minutes. The grids were washed and transferred to 50 μL of uranyl-oxalate solution, pH 7, for 5 minutes, then to a 50 μL drop of methyl-cellulose-UA (a mixture of 4% uranyl acetate and 2% methyl cellulose in a ratio of 100 μL/900 μL, respectively) for 10 minutes on ice. The grids were removed and dried before being observed with JEM 1,011 transmission electron microscope at 80 kV.

Tunable Resistive Pulse Sensing (TRPS)

Exosome concentration and size was analysed with TRPS (qNano, Izon Science Ltd) using a NP100 nanopore at a 45 mm stretch. Exosome concentration and size was standardized using multi-pressure calibration with 70 nm carboxylated polystyrene beads at a known concentration.

Western Blotting

The following antibodies were used for Western blotting: TSG101 (Santa Cruz, sc-6037), CD63 (Abcam, ab8219), Flotillin-1 (BD Transduction Laboratories, 610821), HSP70 (Transduction Laboratories, 610608), Calnexin (Cell Signaling Technology, 2679S), VCP (Abcam, ab11433), GANAB (Abcam, ab179805). Horseradish peroxidase (HRP) conjugated secondary antibodies were purchased from Thermo Scientific. Samples were lysed in reducing sample buffer [0.25 M Tris HCl (pH 6.8), 40% glycerol, 8% SDS, 5% 2-mercaptoethanol and 0.04% bromophenol blue] or non-reducing sample buffer (without 2-mercaptoethanol) and boiled for 10 minutes at 95° C. Proteins were resolved by SDS-PAGE and transferred to polyvinylidene fluoride membranes, blocked in 5% non-fat powdered milk in PBS-T (0.5% Tween-20) and probed with antibodies. Proteins were detected using X-ray film and enhanced chemiluminescence reagent (Amersham ECL Select).

ELISAs

Duoset ELISAs were purchased from R & D systems and used according to manufacturer's instructions. Briefly, capture antibody was diluted to the working concentration in PBS and placed in a 96-well microplate overnight at room temperature. The capture antibody was then removed and the plates washed with wash buffer 3 times. Plates were then blocked with reagent diluent for 2 hours before being washed 3 times with wash buffer. Standards and samples were then incubated for 2 hours in plates before being washed as before. Plates were then incubated with detection antibody for 2 hours and then washed as before. Streptavidin-HRP as then added for 20 minutes, and plates subsequently washed again. Colour was developed by the addition of substrate solution for 20 minutes, before the reaction was stopped by the addition of stop solution. The optical density of each well was determined with a microplate reader set at 450 nm, and wavelength correction at 540 nm.

TNC ELISA kit was purchased from RayBiotech and used according to manufacturer's instructions.

Plasma

Plasma was thawed on ice and centrifuged at 1,500 g for 10 minutes at 4° C. The supernatant was removed, and large vesicles were further removed with another centrifugation step at 10,000 g for 20 minutes at 4° C. 500 μL was then overlaid on qEV size exclusion columns (Izon) followed by elution with PBS. Exosome positive fractions were pooled and concentrated in Amicon®Ultra-4 10 kDa centrifugal filter units to a final volume of 50-100 μL.

Mass Spectrometry

Protein from disrupted exosomes was subjected to proteolytic digestion and analysed on LTQ-OrbitrapElite instrument combined with a Waters NanoAcquity UltraHighPressure Liquid Chromatograph. The number of identifiably discrete proteins within different exosomes on a quantitative basis was processed via a number of purpose-specific software packages

Statistical Analysis

GraphPad Prism version 6.0 and MedCalc version 16.8.4 were used for all calculations. Unpaired Student's t-test was used to calculate the difference in expression values of proteins from exosomes. Receiver operator characteristic (ROC) curves were used to determine the sensitivity and specificity of predictive values. Threshold values were selected using the Youden index. Univariate analysis using the log-rank test was used to assess disease-free survival (Kaplan-Meier curves).

Results

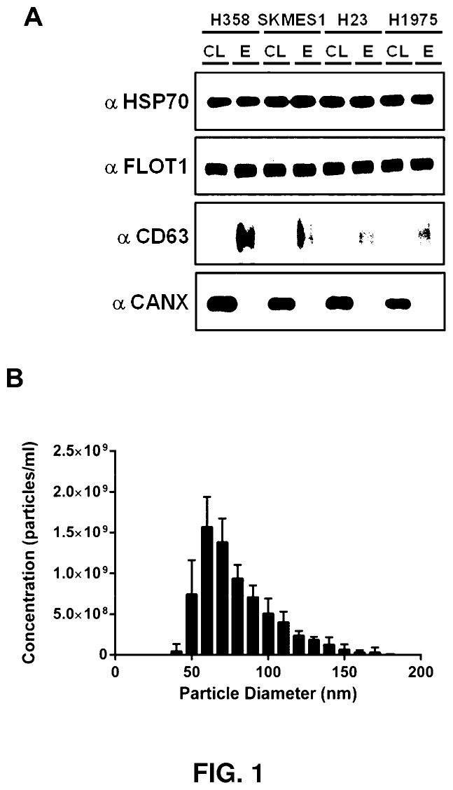

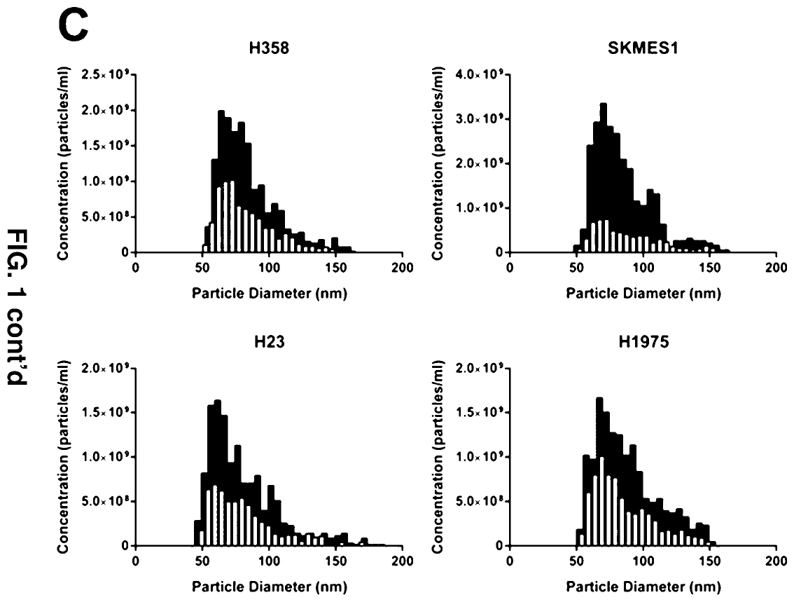

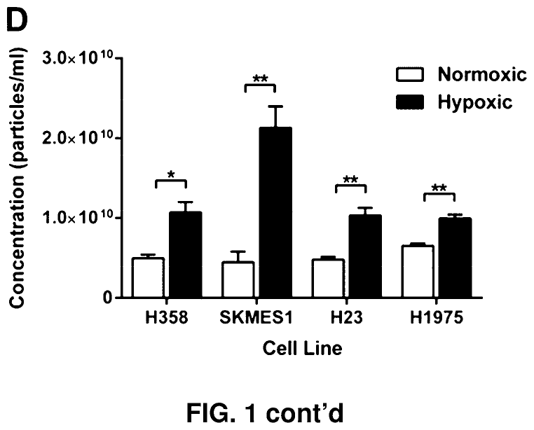

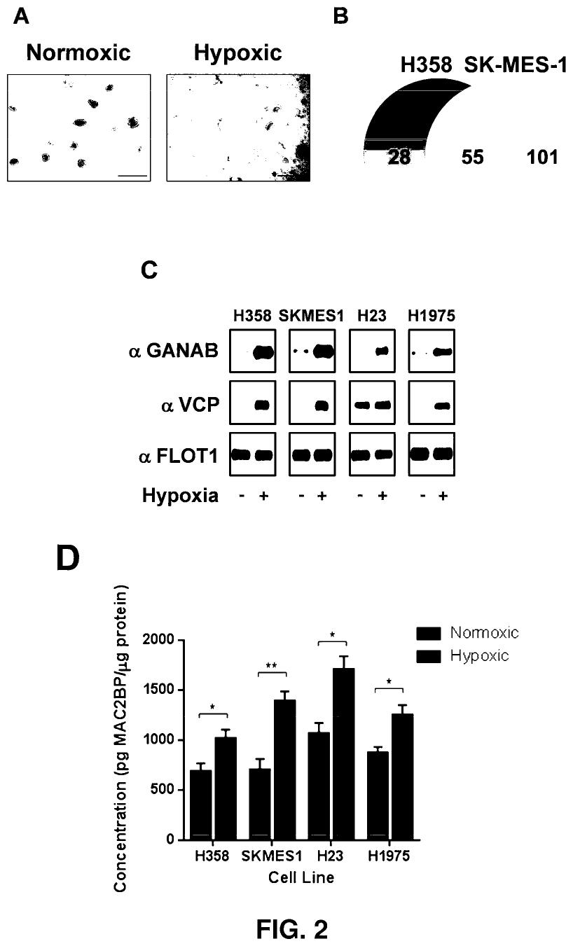

The present study first demonstrated that exosomes were secreted by the NSCLC cell lines H358, SKMES1, H23 and H1975. FIG. 1a shows the presence of canonical exosome proteins and the absence of the endoplasmic reticulum protein Calnexin from exosomes isolated using the above protocol. Furthermore, isolated exosomes exhibit expected morphology and size profiles consistent with pure exosome preparations (FIGS. 1b and 2a).

These NSCLC cell lines were then cultured under hypoxic conditions and the effect on exosome secretion was monitored. As can be observed in FIGS. 1c, 1d and 2a, hypoxic conditions induced the secretion of exosomes from each of the four cell lines investigated, but the range of exosome size and morphology was unchanged.

The present study then sought to determine whether the hypoxia modified the protein content or signatures of the exosomes secreted by the NSCLC cell lines. Quantitative mass spectrometry demonstrated that exosomes from the H358 and SKMES-1 cell lines had a respective 83 and 156 upregulated proteins with hypoxia, of which a total of 55 upregulated proteins were common to both cell lines (FIG. 2b, Table 1). The present study then sought to validate this mass spectrometry data. To this end, two of the upregulated proteins identified by mass spectrometry, namely Neutral alpha-glucosidase AB (GANAB) and Transitional endoplasmic reticulum ATPase (VCP), were shown to be upregulated in hypoxic exosomes from the four NSCLC cell lines by western blot and ELISA (FIGS. 2c and 2d), thereby supporting the mass spectrometry data.

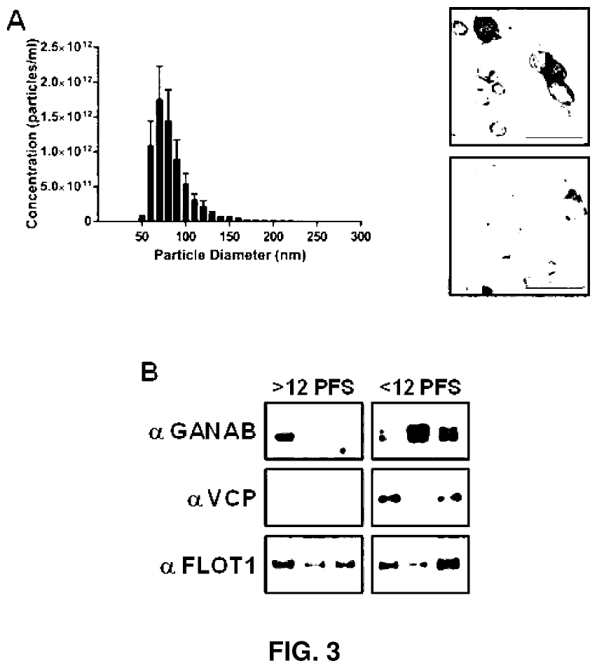

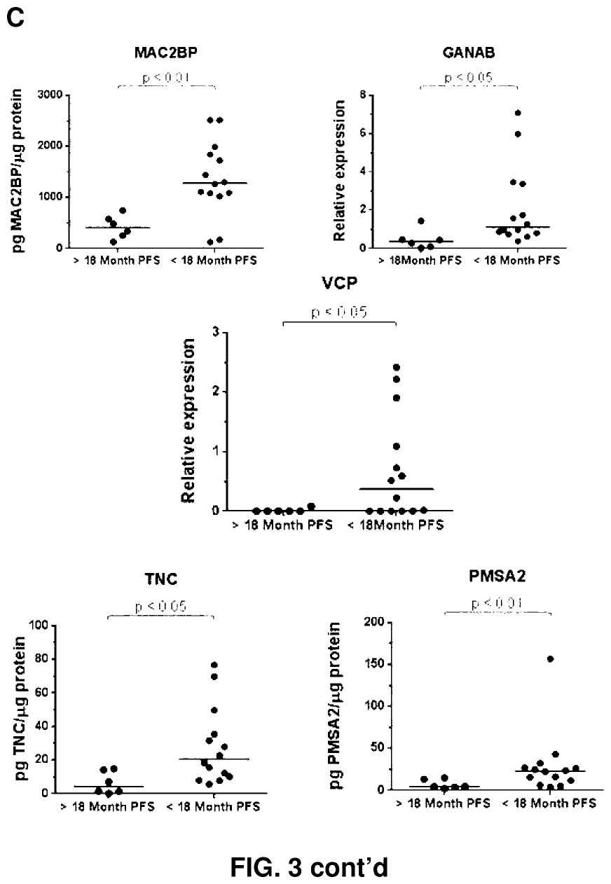

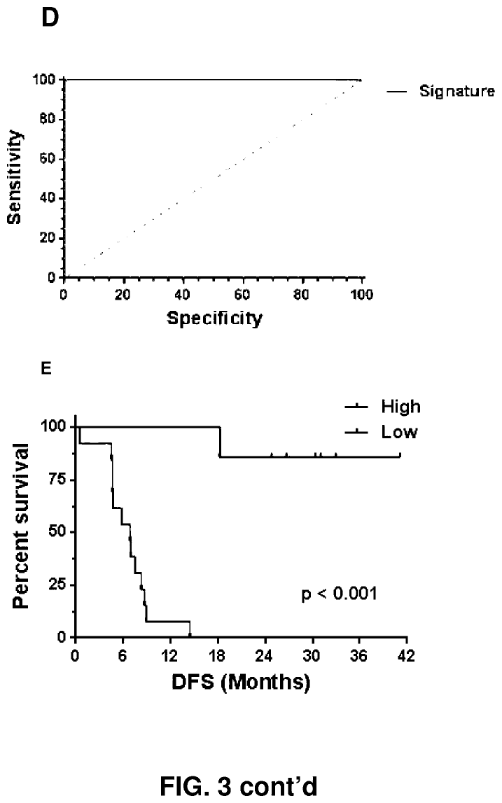

The present study then sought to determine whether these proteins upregulated with hypoxia correlated to patient disease progression in NSCLC. As can be seen in FIG. 3a, exosomes isolated from the plasma of NSCLC patients demonstrate a typical size range and morphology. It was then demonstrated by western blot that the hypoxic exosomal protein markers of GANAB, VCP, Galectin-3-Binding Protein, TNC and PMSA2 were significantly upregulated in NSCLC patients with a poorer prognosis (i.e., those that progress or relapse within the first 12 months after treatment) (FIG. 3c). The ROC curve in FIG. 3d further demonstrates that the combined protein signature of GANAB, VCP and Galectin-3-Binding Protein has a high overall accuracy with respect to identifying NSCLC patients of a poor prognosis. This is supported by FIG. 3e that reveals that NSCLC patients with upregulated exosomal expression of at least 2 of the GANAB, VCP and Galectin-3-Binding Protein proteins demonstrate a significantly shorter period of disease-free survival than those patients with only one or none of these markers highly expressed in their exosomes.

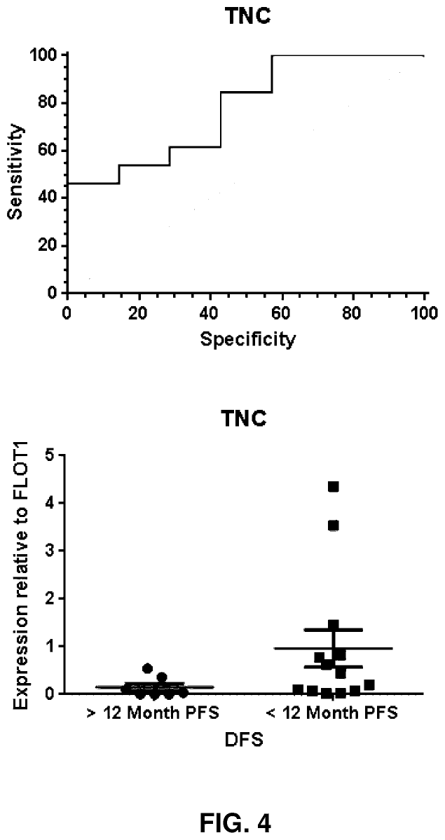

In addition to the exosomal protein markers of GANAB, VCP and Galectin-3-Binding Protein, additional proteins from the original 55 hypoxia protein signature identified in NSCLC cell lines may also be of prognostic value. For example, FIG. 4 demonstrates that Tenascin C (TNC) protein levels is also upregulated in the exosomes of NSCLC patients more likely to progress following treatment. Additionally, the ROC curve in FIG. 4 demonstrates that on its own demonstrates considerable accuracy with respect to identifying NSCLC patients of a poor prognosis.

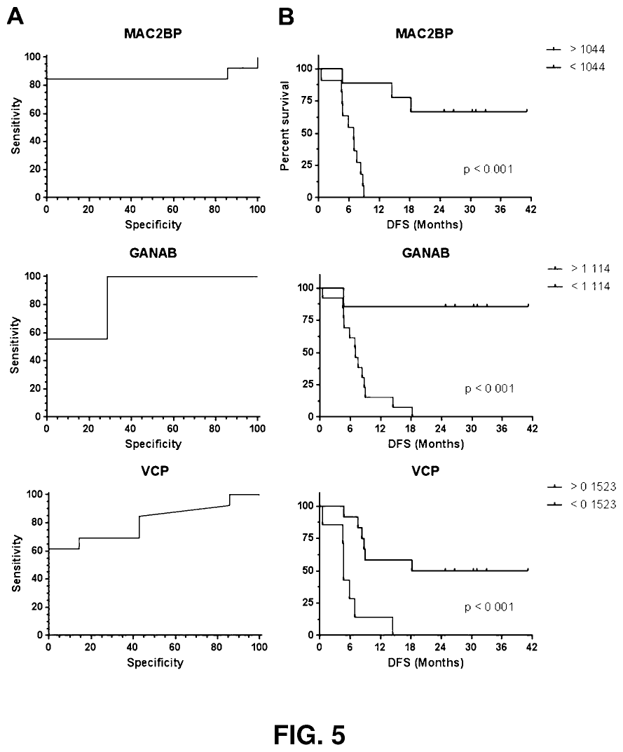

Individual protein ROC and survival curves for that data with respect to the patient exosomal proteins of GANAB, VCP and Galectin-3-Binding Protein (MAC2BP) demonstrated in FIGS. 3d and 3e are provided in FIG. 5. This data confirms that even on their own, each of these 3 proteins are accurate prognostic markers with respect to disease progression in NSCLC patients.

### CONCLUSION

These data indicate that the above protein markers identified in hypoxic exosomes in vitro represent potential prognostic biomarkers for disease progression or relapse in NSCLC cancer patients. Accordingly, such exosomal biomarkers may represent reliable and non-invasive prognostic markers for a variety of solid malignancies.

TABLE 1
Upregulated proteins common to H358 and SKMES-1 cell lines.

| Protein name | Accession No. |
|---|---|
| 40S ribosomal protein S15 | P62841 |
| 60 kDa heat shock protein, mitochondrial | P10809 |
| Afadin | P55196 |
| Agrin | O00468 |
| Amyloid beta A4 protein | P05067 |
| Aspartyl aminopeptidase | Q9ULA0 |
| ATP-citrate synthase | P53396 |
| Calsyntenin-1 | O94985 |
| Complement factor H | P08603 |
| Cullin-associated NEDD8-dissociated protein 1 | Q86VP6 |
| Fatty acid synthase | P49327 |
| Filamin-A | P21333 |
| Filamin-B | O75369 |
| Fructose-bisphosphate aldolase A | P04075 |
| Galectin-3-binding protein | Q08380 |
| Glutamate dehydrogenase 1, mitochondrial | P00367 |
| Laminin subunit alpha-3 | Q16787 |
| Laminin subunit alpha-5 | O15230 |
| Laminin subunit beta-1 | P07942 |
| Laminin subunit beta-2 | P55268 |
| Laminin subunit gamma-1 | P11047 |
| Lysyl oxidase homolog 2 | Q9Y4K0 |
| MIT domain-containing protein 1 | Q8WV92 |
| Neutral alpha-glucosidase AB | Q14697 |
| Nucleolar protein 56 | O00567 |
| Prolow-density lipoprotein receptor-related protein 1 | Q07954 |
| Proteasome activator complex subunit 1 | Q06323 |
| Proteasome subunit alpha type-1 | P25786 |
| Proteasome subunit alpha type-2 | P25787 |
| Proteasome subunit alpha type-3 | P25788 |
| Proteasome subunit alpha type-4 | P25789 |
| Proteasome subunit alpha type-5 | P28066 |
| Proteasome subunit alpha type-6 | P60900 |
| Proteasome subunit beta type-1 | P20618 |
| Proteasome subunit beta type-2 | P49721 |
| Proteasome subunit beta type-3 | P49720 |
| Proteasome subunit beta type-4 | P28070 |
| Proteasome subunit beta type-5 | P28074 |
| Proteasome subunit beta type-6 | P28072 |
| Proteasome subunit beta type-7 | Q99436 |
| Proteasome subunit beta type-8 | P28062 |
| Protein arginine N-methyltransferase 5 | O14744 |
| Protein LAP2 | Q96RT1 |
| Proto-oncogene tyrosine-protein kinase Src | P12931 |
| Serine incorporator 5 | Q86VE9 |
| Spectrin alpha chain, non-erythrocytic 1 | Q13813 |
| Spectrin beta chain, non-erythrocytic 1 | Q01082 |
| Splicing factor 3B subunit 3 | Q15393 |
| Syntaxin-binding protein 2 | Q15833 |
| Tenascin | P24821 |
| Tensin-3 | Q68CZ2 |
| Thrombospondin-1 | P07996 |
| Transitional endoplasmic reticulum ATPase | P55072 |
| Translational activator GCN1 | Q92616 |
| UDP-glucuronic acid decarboxylase 1 | Q8NBZ7 |

### REFERENCES

1. Thun M J, Lally C A, Flannery J T, Calle E E, Flanders W D, Heath C W, Jr. Cigarette smoking and changes in the histopathology of lung cancer [see comments]. J Natl Cancer Inst 1997; 89:1580-6.
2. Mathers C, Vos T. The burden of disease and injury in Australia-summary report. Canberra: Australian Institute of Health and Welfare ISBN 1 74024 022 7; 1999.
3. Larsen J E, Pavey S J, Bowman R, et al. Gene expression of lung squamous cell carcinoma reflects mode of lymph node involvement. Eur Respir J 2007; 30:21-5.
4. Larsen J E, Pavey S J, Passmore L H, et al. Expression profiling defines a recurrence signature in lung squamous cell carcinoma. Carcinogenesis 2007; 28:760-6.
5. Larsen J E, Pavey S J, Passmore L H, Bowman R V, Hayward N K, Fong K M. Gene expression signature predicts recurrence in lung adenocarcinoma. Clin Cancer Res 2007; 13:2946-54.
6. S ELA, Mager I, Breakefield X O, Wood M J. Extracellular vesicles: biology and emerging therapeutic opportunities. Nat Rev Drug Discov 2013; 12:347-57.
7. Valadi H, Ekstrom K, Bossios A, Sjostrand M, Lee J J, Lotvall J O. Exosome-mediated transfer of mRNAs and microRNAs is a novel mechanism of genetic exchange between cells. Nature cell biology 2007; 9:654-9.
8. Sceneay J, Chow M T, Chen A, et al. Primary tumor hypoxia recruits CD11b+/Ly6Cmed/Ly6G+ immune suppressor cells and compromises N K cell cytotoxicity in the premetastatic niche. Cancer Res 2012; 72:3906-11.
9. Chafe S C, Lou Y, Sceneay J, et al. Carbonic anhydrase IX promotes myeloid-derived suppressor cell mobilization and establishment of a metastatic niche by stimulating G-CSF production. Cancer Res 2015; 75:996-1008.
10. Martins V R, Dias M S, Hainaut P. Tumor-cell-derived microvesicles as carriers of molecular information in cancer. Curr Opin Oncol 2013; 25:66-75.
11. Kucharzewska P, Christianson H C, Welch J E, et al. Exosomes reflect the hypoxic status of glioma cells and mediate hypoxia-dependent activation of vascular cells during tumor development. Proc Natl Acad Sci USA 2013; 110:7312-7.
12. Xiang X, Poliakov A, Liu C, et al. Induction of myeloid-derived suppressor cells by tumor exosomes. Int J Cancer 2009; 124:2621-33.
13. Al-Nedawi K, Meehan B, Kerbel R S, Allison A C, Rak J. Endothelial expression of autocrine VEGF upon the uptake of tumor-derived microvesicles containing oncogenic EGFR. Proc Natl Acad Sci USA 2009; 106:3794-9.
14. Al-Nedawi K, Meehan B, Micallef J, et al. Intercellular transfer of the oncogenic receptor EGFRvIII by microvesicles derived from tumour cells. Nat Cell

Biol 2008; 10:619-24.
15. Ciravolo V, Huber V, Ghedini G C, et al. Potential role of HER2-overexpressing exosomes in countering trastuzumab-based therapy. J Cell Physiol 2012; 227:658-67.
16. Peinado H, Aleckovic M, Lavotshkin S, et al. Melanoma exosomes educate bone marrow progenitor cells toward a pro-metastatic phenotype through MET. Nature medicine 2012; 18:883-91.
17. Demory Beckler M, Higginbotham J N, Franklin J L, et al. Proteomic analysis of exosomes from mutant KRAS colon cancer cells identifies intercellular transfer of mutant KRAS. Mol Cell Proteomics 2013; 12:343-55.
18. Corcoran C, Rani S, O'Brien K, et al. Docetaxel-resistance in prostate cancer: evaluating associated phenotypic changes and potential for resistance transfer via exosomes. PLoS One 2012; 7:e50999.
19. Wysoczynski M, Ratajczak M Z. Lung cancer secreted microvesicles: underappreciated modulators of microenvironment in expanding tumors. Int J Cancer 2009; 125:1595-603.
20. Lv L H, Wan Y L, Lin Y, et al. Anticancer drugs cause release of exosomes with heat shock proteins from human hepatocellular carcinoma cells that elicit effective natural killer cell antitumor responses in vitro. J Biol Chem 2012; 287:15874-85.
21. Khan S, Jutzy J M, Aspe J R, McGregor D W, Neidigh J W, Wall N R. Survivin is released from cancer cells via exosomes. Apoptosis 2011; 16:1-12.
22. Safaei R, Larson B J, Cheng T C, et al. Abnormal lysosomal trafficking and enhanced exosomal export of cisplatin in drug-resistant human ovarian carcinoma cells. Mol Cancer Ther 2005; 4:1595-604.
23. Vlassov A V, Magdaleno S, Setterquist R, Conrad R. Exosomes: current knowledge of their composition, biological functions, and diagnostic and therapeutic potentials. Biochim Biophys Acta 2012; 1820:940-8.
24. Melo S A, Luecke L B, Kahlert C, et al. Glypican-1 identifies cancer exosomes and detects early pancreatic cancer. Nature 2015; 523:177-82.
25. Thakur B K, Zhang H, Becker A, et al. Double-stranded DNA in exosomes: a novel biomarker in cancer detection. Cell research 2014; 24:766-9.
26. Kahlert C, Melo S A, Protopopov A, et al. Identification of double-stranded genomic DNA spanning all chromosomes with mutated KRAS and p53 DNA in the serum exosomes of patients with pancreatic cancer. J Biol Chem 2014; 289:3869-75.
27. Roberson C D, Atay S, Gercel-Taylor C, Taylor D D. Tumor-derived exosomes as mediators of disease and potential diagnostic biomarkers. Cancer biomarkers: section A of Disease markers 2010; 8:281-91.
28. Zocco D, Ferruzzi P, Cappello F, Kuo W P, Fais S. Extracellular vesicles as shuttles of tumor biomarkers and anti-tumor drugs. Frontiers in oncology 2014; 4:267.
29. Wen S W, Everitt S J, Bedo J, et al. Spleen Volume Variation in Patients with Locally Advanced Non-Small Cell Lung Cancer Receiving Platinum-Based Chemo-Radiotherapy. PLoS One 2015; 10:e0142608.
30. Lobb R J, Becker M, Wen S W, et al. Optimized exosome isolation protocol for cell culture supernatant and human plasma. Journal of extracellular vesicles 2015; 4:27031.

### Example 2

Despite significant therapeutic advances, lung cancer remains the leading cause of cancer-related death worldwide. Non-small cell lung cancer (NSCLC) patients have a very poor overall five-year survival rate as low as 15%². Biopsies are used to diagnose and subtype NSCLC, and TNM staging is the most important factor for predicting survival and guiding clinical interventions². However, a significant proportion of early stage and locoregionally-confined NSCLC patients have therapy-refractory disease or develop metastatic disease despite curative intent treatment with surgery radiotherapy or chemoradiotherapy, demonstrating that TNM staging alone is insufficient in guiding disease management. Therefore, there is a significant unmet clinical need to identify these patients who respond poorly to current treatments and would allow for a tailoring of treatment interventions. Prognostic biomarkers—in particular non-invasive liquid biomarkers—could allow clinicians to triage patients who require intensification of treatment or adjuvant treatment interventions.

Small extracellular vesicles, termed exosomes, have been shown to serve as a non-invasive method for identifying outcome in pancreatic cancer³. Exosomes are secreted, membrane enclosed vesicles with a size-range of 30-150 nm in diameter⁴. Originating from the inward budding of multivesicular bodies, exosomes contain a variety of nucleic acids, lipids and proteins derived from their cell of origin⁴. Upon fusion with the plasma membrane, exosomes are released into the extracellular environment and capable of entering the circulation⁴. It is for this reason that exosome isolation from the body fluids of patients serves as a potential source of novel markers that can serve to characterise NSCLC in more detail compared with currently available clinical techniques.

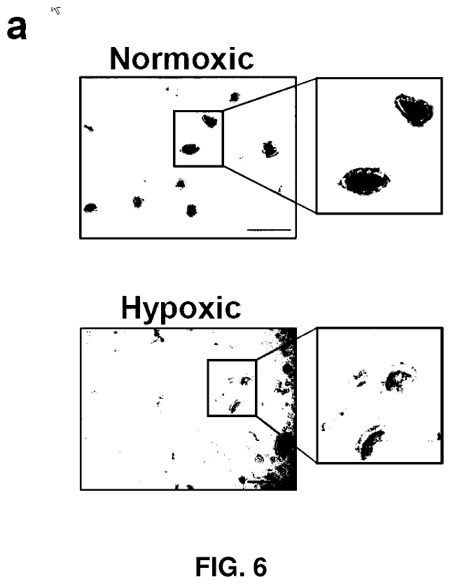

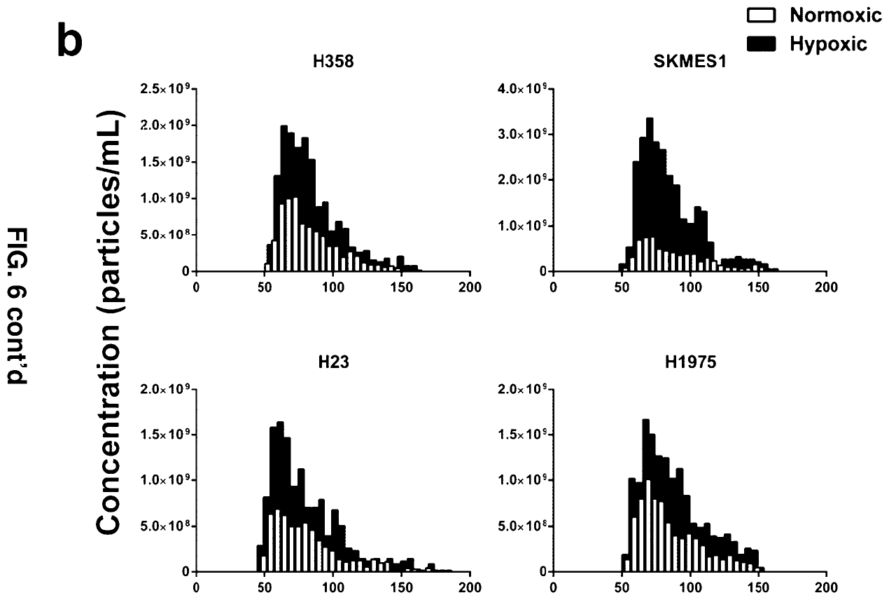

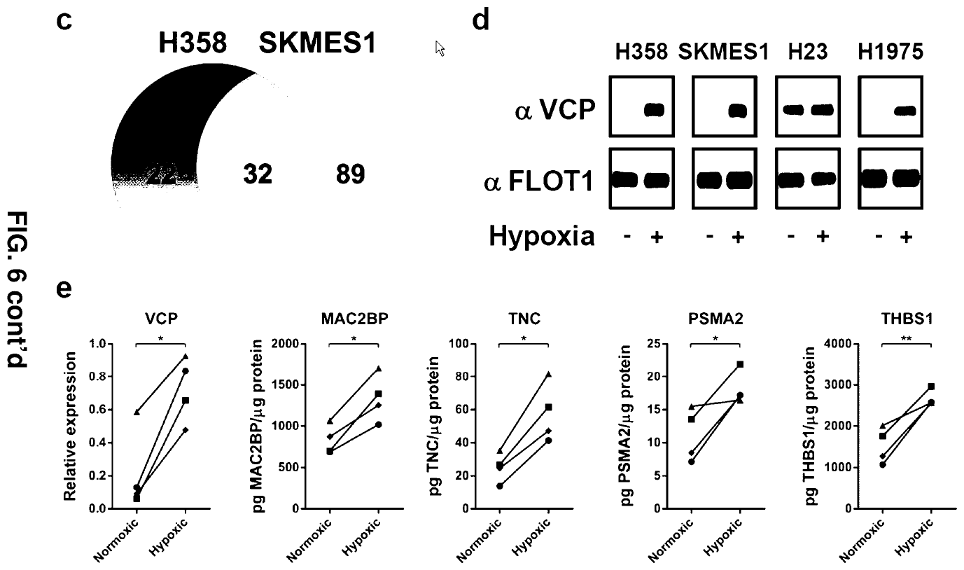

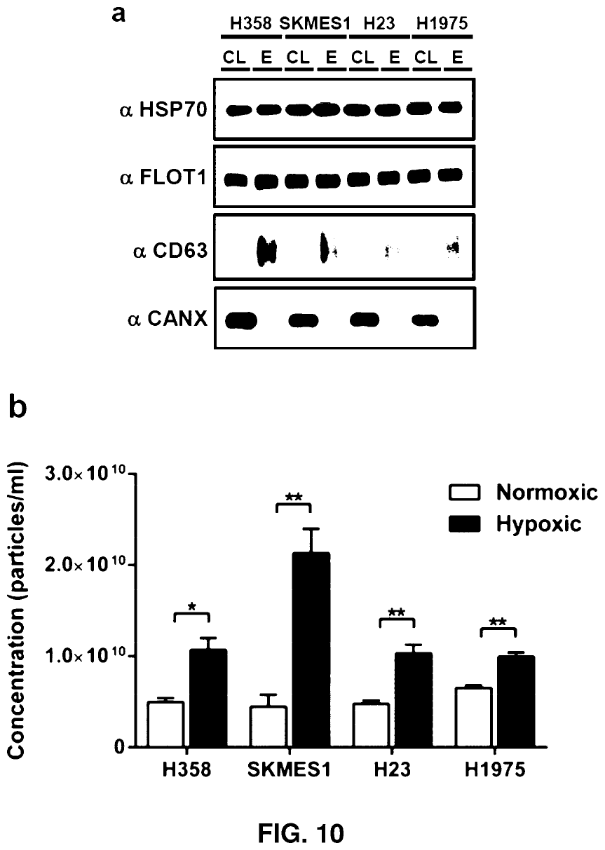

It is well established that hypoxia occurs early during tumour development and causes an aggressive, invasive and metastatic phenotype^{5,6}. We postulated that NSCLC cells exposed to hypoxic conditions would secrete exosomes with a distinct proteome profile, indicative of an aggressive phenotype of the cell of origin. To address if hypoxia causes changes to exosomal protein content, we isolated exosomes secreted by human NSCLC lines (H358, SKMES1, H23, and H1975) cultured under normoxic (21% O₂), or hypoxic (2% O₂) conditions (FIGS. 6A & B and FIG. 10) using established methods^{7,8}. Exosomes displayed typical size distribution when measured by tunable resistive pulse sensing (TRPS), and contained canonical exosome markers HSP70, FLOT1 and CD63 (FIG. 6B; FIG. 10A). Interestingly, transmission electron microscopy (TEM) and TRPS nanoparticle analysis revealed NSCLC cells significantly increased exosome secretion in response to hypoxia (FIGS. 6A & B; FIG. 10B). The proteomes of normoxic and hypoxia-derived exosomes from the adenocarcinoma H358 and squamous cell carcinoma SKMES1 cells were evaluated using mass spectrometry. Label-free quantification by spectral counting identified 32 proteins that were unregulated under low oxygen tension in both H358 and SKMES1 exosomes (16 cytoplasmic, 10 secreted, and 6 transmembrane) (FIG. 6C; Tables 2 & 3). Based on the previous association with to cancer progression, an exosome signature based on 5 of these proteins (2 cytoplasmic [VCP⁹, PSMA2¹⁰], 2 secreted [TNC^{11,12}, THBS1¹³], and 1 transmembrane protein [MAC2BP¹⁴]) was selected. All 5 proteins were confirmed to be contained at higher abundances in exosomes derived from additional hypoxic NSCLC cell lines (FIGS. 1 D & E).

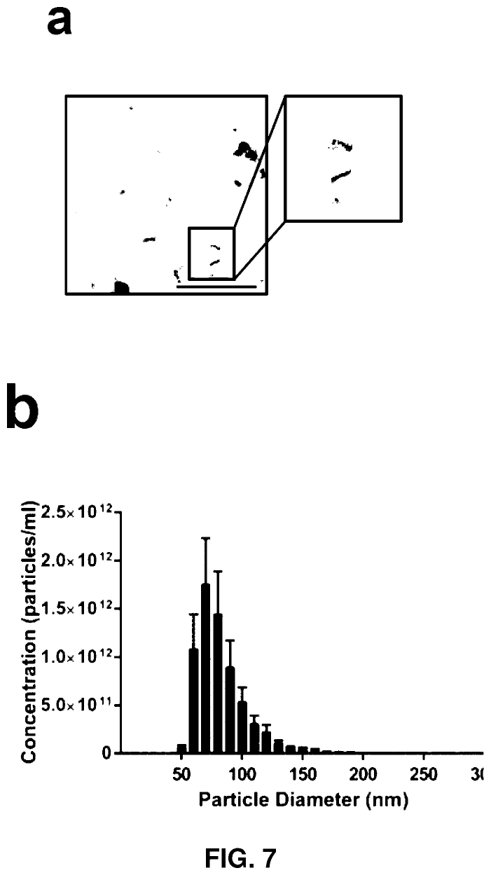

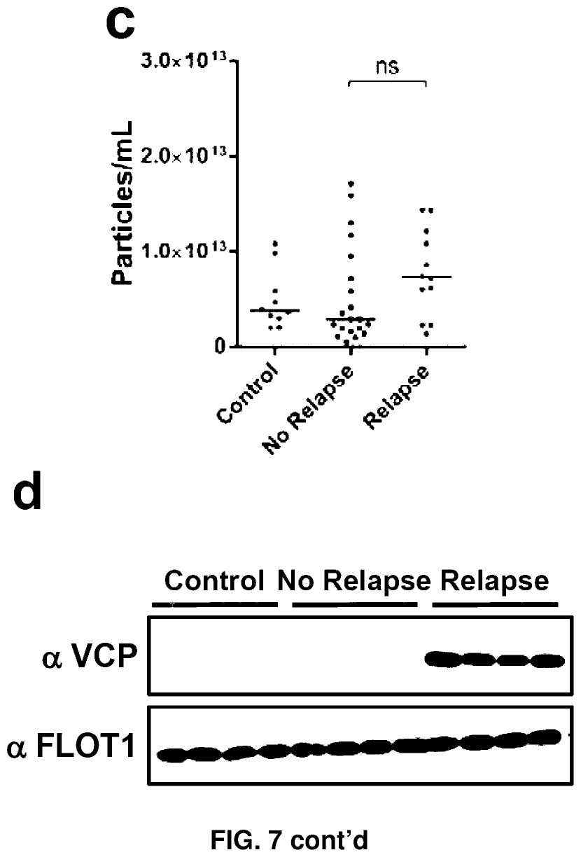

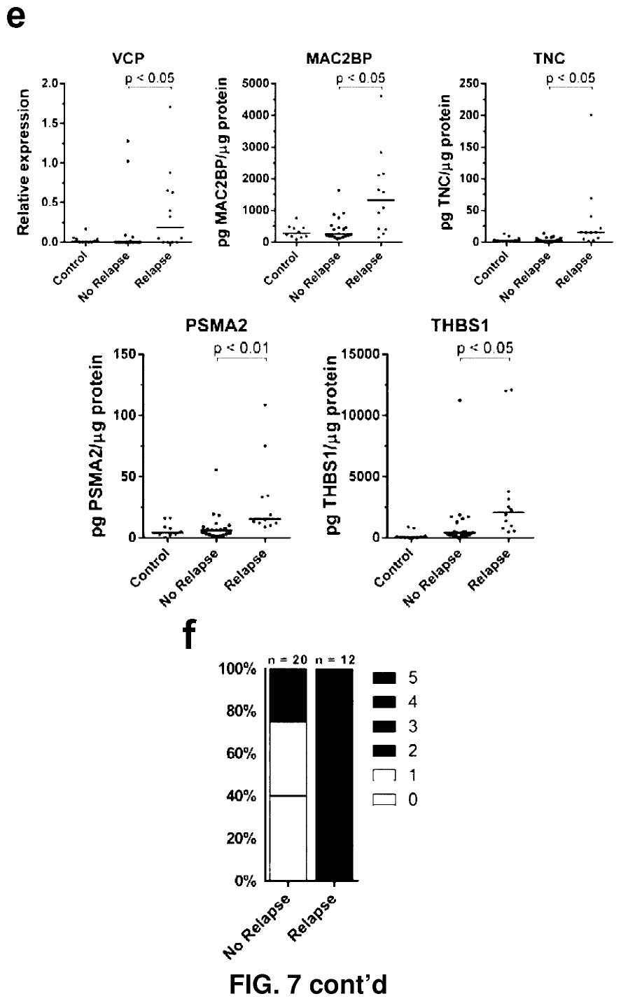

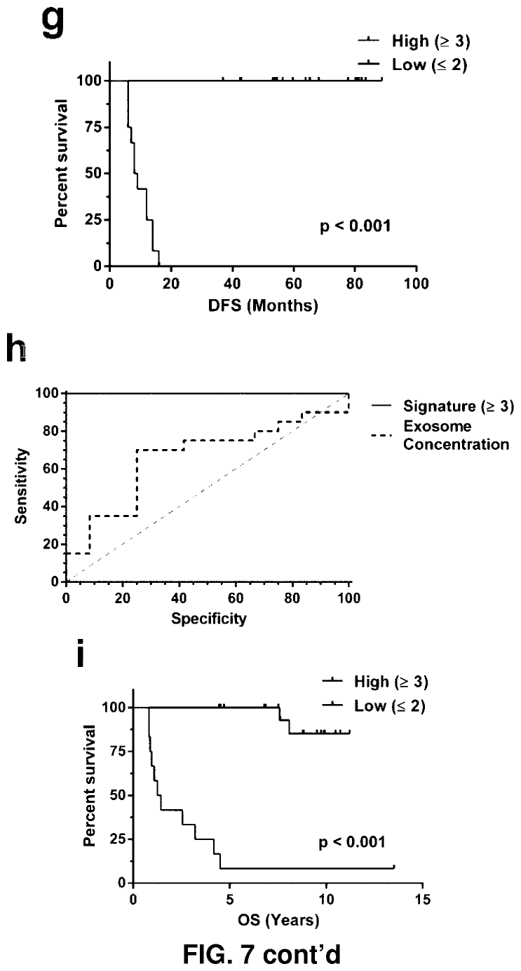

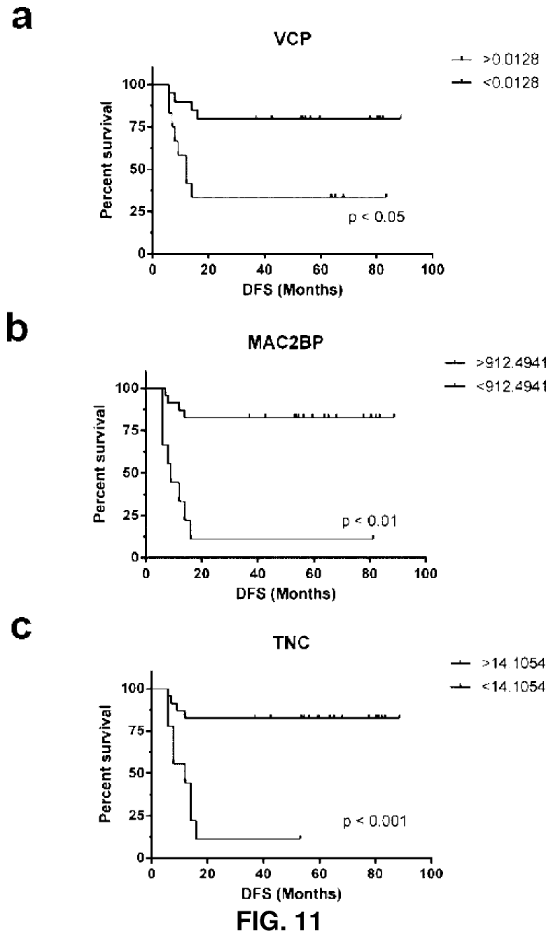

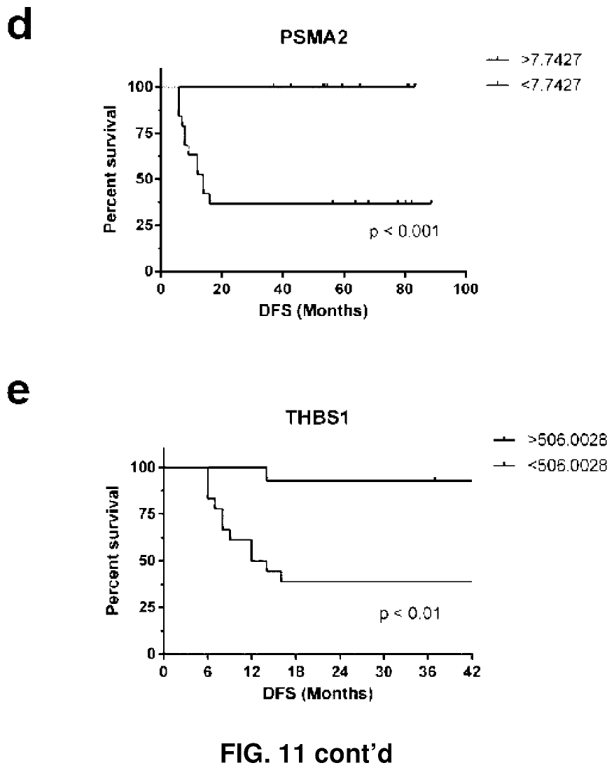

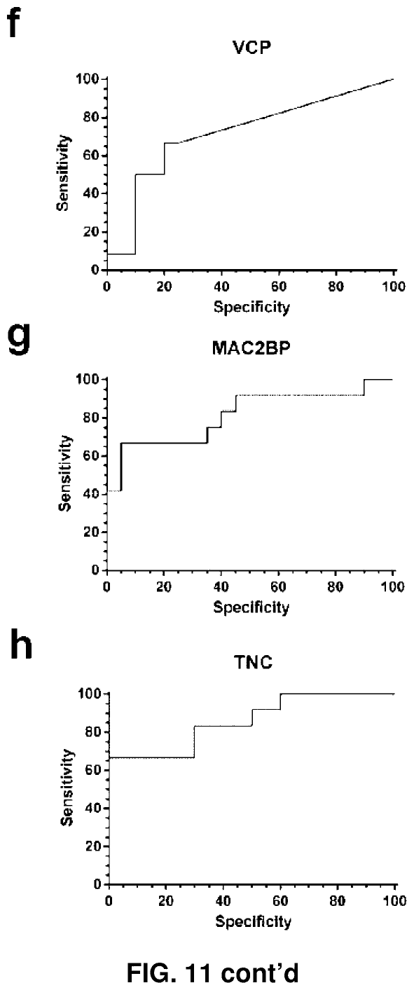

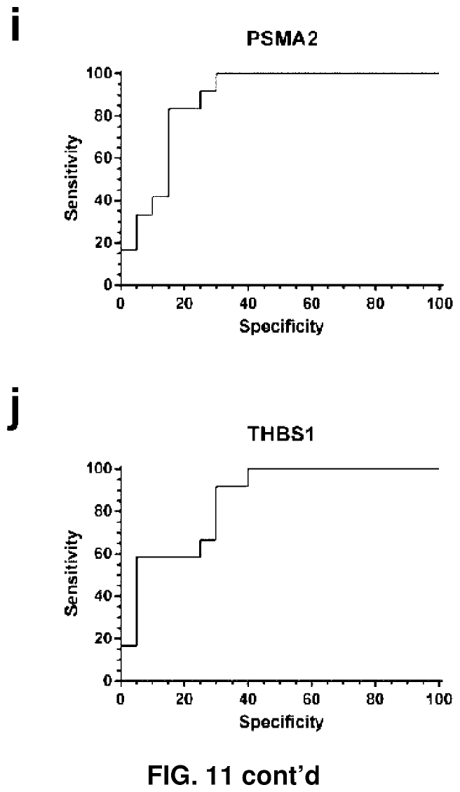

We then postulated that hypoxic-induced exosomal changes could be utilised as a prognostic biomarker for disease progression in early-stage NSCLC. Exosomes were isolated from the plasma of a 32 patient treatment naive stage I-III NSCLC discovery cohort sampled at the time of diagnosis (FIGS. 7A & B). Although hypoxia increases exosome secretion from NSCLC cells (FIG. 10B), we surprisingly found that exosome concentration in the plasma of NSCLC patients had no prognostic value for clinical relapse within 18 months as a categorical variable (FIG. 7C). Interestingly, the combined 5 protein exosome signature (VCP, MAC2BP, TNC, PSMA2, and THBS1) was specifically increased in exosomes derived from NSCLC subjects who relapsed (FIG. 7D). Each protein from the exosome signature was individually an excellent prognostic biomarker of disease relapse (FIG. 11). Interestingly, we were able to generate a clear separation in the disease-free survival (DFS) of patients based on the abundance of these 5 exosomal proteins that exceeded Youden's threshold value (≤2=No relapse; ≥3=Relapse) (FIGS. 7F & G). Importantly, the receiver operating characteristic (ROC) curve demonstrates that these 5 exosomal proteins have the capacity to prognosticate disease progression at 100% specificity and sensitivity (FIG. 7F) within this discovery cohort. Moreover, the exosome signature was capable of separating patients overall survival (OS) in the discovery cohort (FIG. 71), indicating that both relapse and OS is linked to the abundance of the exosome signature.

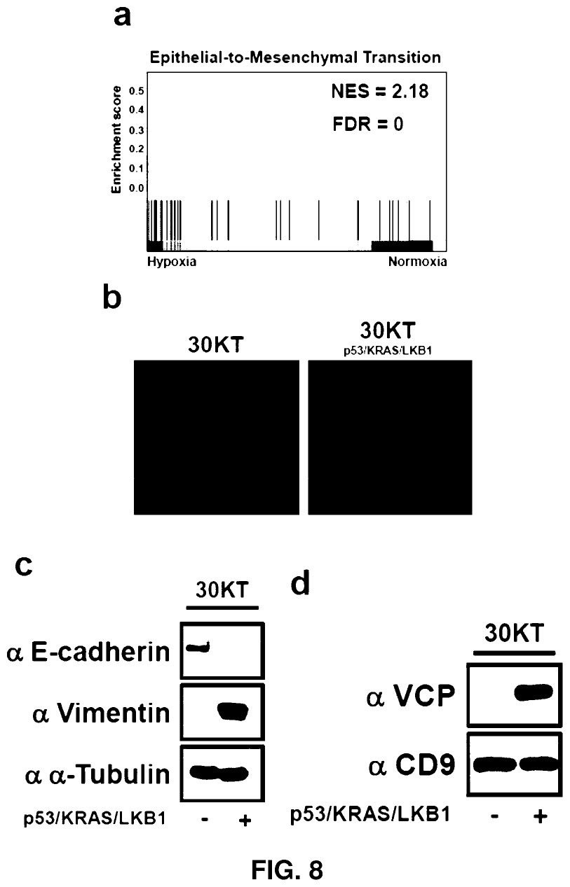

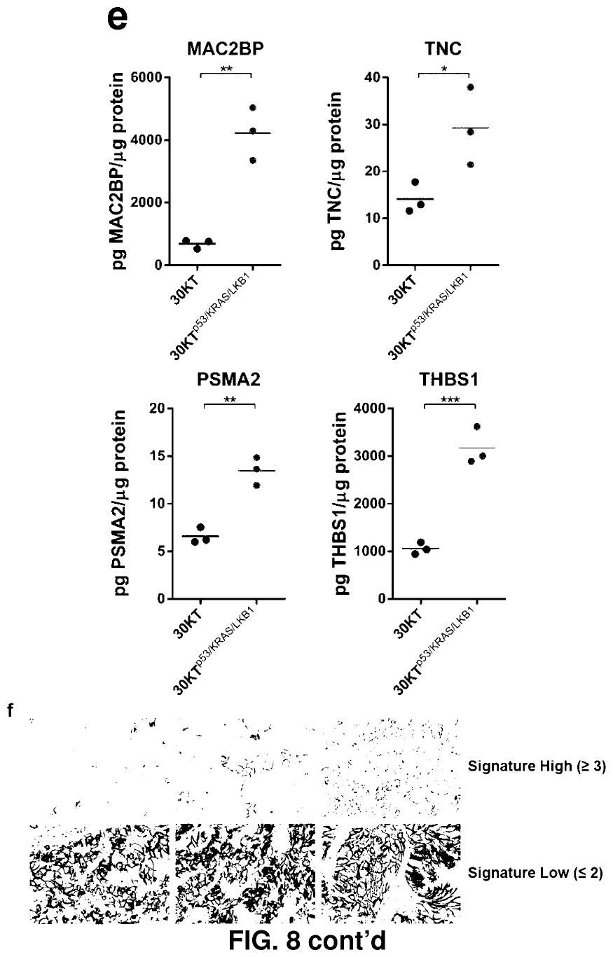

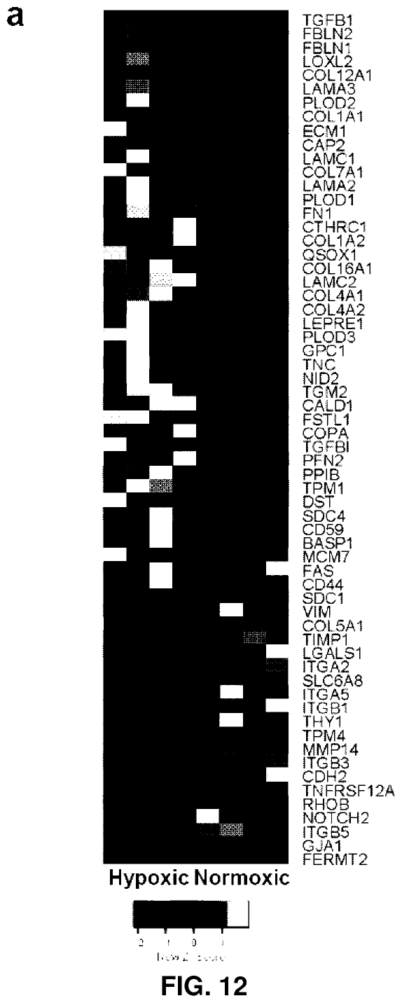

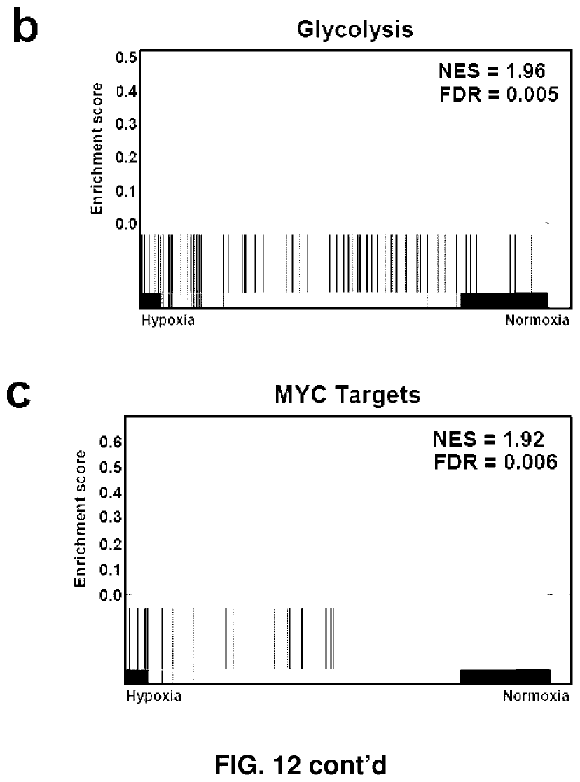

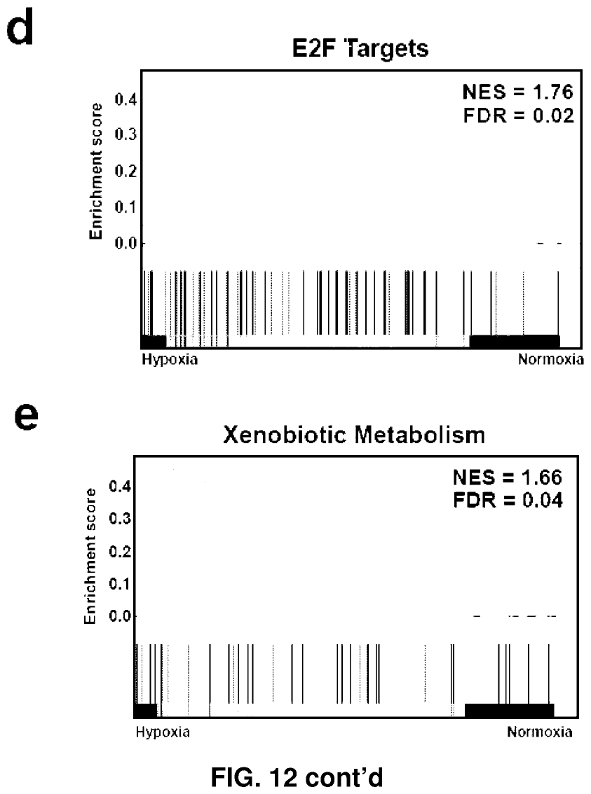

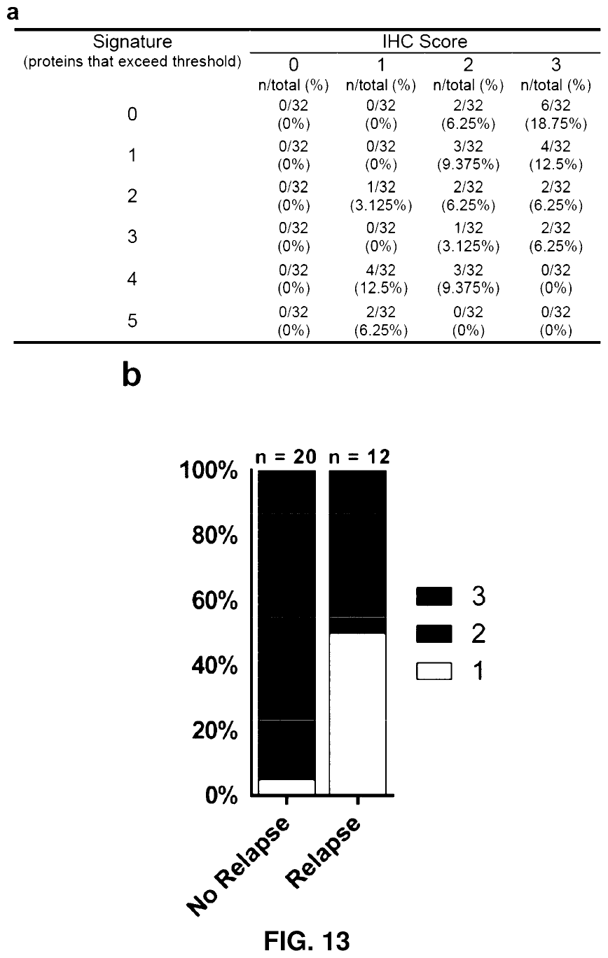

On the basis of the prognostic value of the exosome signature we investigated the potential mechanism underpinning this exosomal signature. We have recently demonstrated the protein content of exosomes can reflect the phenotype of the cell of origin¹⁵, we performed gene set enrichment analysis (GSEA) on total protein abundance in exosomes derived from normoxic or hypoxic conditions. A number of gene sets were significantly enriched in NSCLC cell-derived exosomes isolated under hypoxic conditions (FIG. 12), including glycolysis, MYC targets, E2F targets, and xenobiotic metabolism. Interestingly, the top ranked gene set enriched in hypoxic exosomes was associated with EMT (FIG. 8A; FIG. 12A). Given that hypoxia is a strong inducer of EMT in cancer cells¹⁶, we postulated that a mesenchymal phenotype alone could be sufficient to cause the exosomal signature secretion. To determine if the 5 exosomal proteins are secreted by normal or transformed lung epithelial cells, we isolated exosomes from an isogenic human bronchial epithelial cell (HBECs) line. Strikingly, HBECs that underwent oncogenically-induced EMT (FIGS. 8B & C) through p53 knockdown, Kras v12 overexpression and LKB1 knockdown (30KT^{p53/KRAS/LKB1})¹⁷, secreted elevated exosomal signature proteins even under normoxic conditions (FIGS. 8D & E). To validate the link of mesenchymal lung cancer cells secreting the exosome signature we then analysed E-cadherin expression in patient tumour biopsies from the discovery cohort. Immunohistochemistry of tumour biopsies revealed a significant correlation (R²=0.458, p<0.001) (FIG. 13) of reduced E-cadherin expression in tumours from patients with a high exosome signature score of ≥3 compared to patients with an exosome signature score of ≤2 (FIG. 8F). These data support the notion that EMT in oncogenically transformed lung cells is causative for the elevated proteins levels found in our exosome signature both in vitro and in vivo in NSCLC patients.

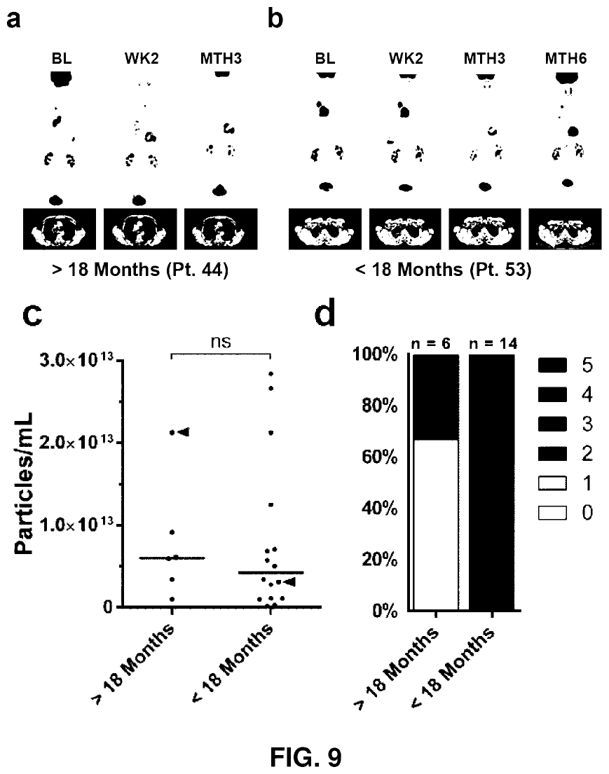

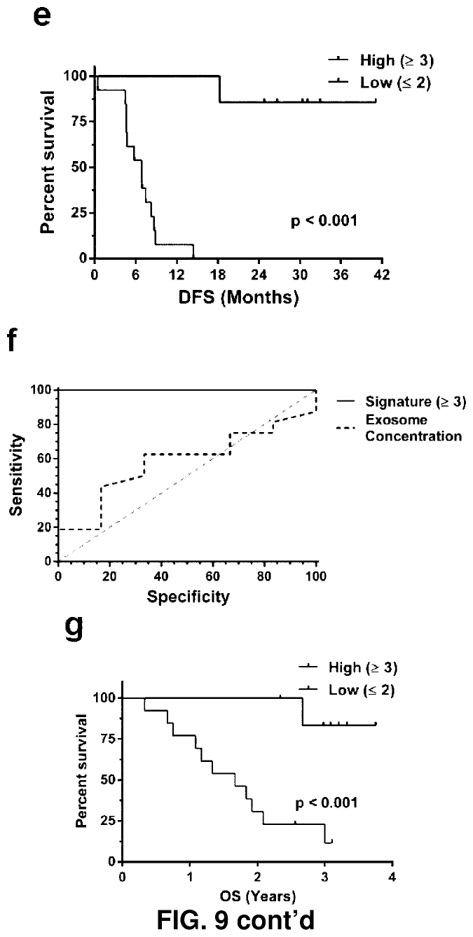

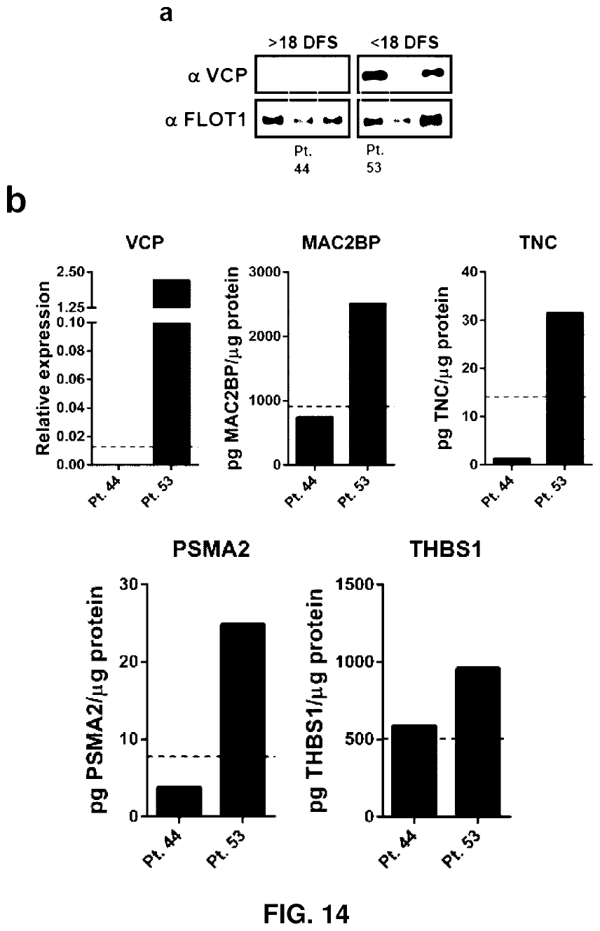

The phenotypic depolarisation of epithelial cells into elongated mesenchymal cells not only promotes an aggressive and metastatic phenotype of cancer cells, but also chemotherapy resistance^{17,18}. Therefore, for independent validation, we evaluated 20 locally advanced NSCLC subjects (confirmation cohort) receiving standard of care chemoradiation, consisting of conformal RT (60 Gy/30 fractions, 6 weeks) with concomitant chemotherapy (either cisplatin/etoposide or carboplatin/paclitaxel). Patients were monitored at baseline, Day 10, Day 24 and Day 90 with ¹⁸F-FDG PET/CT and with standard CT-scan at three monthly intervals for 12 months and six monthly intervals thereafter (FIGS. 9A & B; Table 5). Exosome concentration was measured at baseline using TRPS. Subjects who relapsed within 18 months had no significant differences in circulating exosome abundance (FIG. 9C). In agreement with the discovery cohort, the exosomal protein signature showed significant elevation and prognostic value in subjects who relapsed within 18 months, compared to those who did not relapse within 18 months (FIG. 9D; FIG. 14). Using the same threshold values and algorithm (≤2 markers=low risk of relapsing within 18 months; ≥3=high risk of relapsing within 18 months) established in the discovery cohort, the signature clearly separated patients that relapsed within 18 months and patients that relapsed after 18 months (FIGS. 9D & E). ROC curve analysis further confirmed the specificity and sensitivity of the exosome signature for disease relapse (FIG. 9F). In further agreement with the discovery cohort, the exosome signature could separate patients on the basis of OS, indicating the exosomal protein signature is an ideal classifier of subjects who relapse early and have poor overall survival.

Given the association of EMT with metastasis and chemoresistance¹⁶⁻²⁰ these data identify a mechanism for the short disease-free survival seen in both cohorts of NSCLC patients in this study. This work demonstrates that hypoxia/EMT-related exosomal biomarkers are very promising for identifying early stage NSCLC patients at risk of early recurrence and poor clinical outcome. Hypoxia has diverse functions in promoting tumour growth and metastasis^{5,6,21}, including the induction of the developmental EMT program¹⁶, thereby promoting metastasis and chemoresistance in cancer cells^{16-20,22}. Importantly, the capability of non-invasively, and reliably, detecting hypoxia and/or EMT in NSCLC may serve as a potential prognostic screening tool in early stage NSCLC, facilitating curative therapies and reducing overall mortality. Our results provide strong initial evidence for a newly discovered exosomal protein signature as a marker of disease progression in NSCLC. Further work will be carried out to determine if the exosome signature is a predictive biomarker in the setting of chemoradiation, or whether the exosome signature is a prognostic biomarker in the setting of NSCLC in general. Although TNM staging provides significant benefit in patient management and will remain key in clinical management of NSCLC patients, the exosome signature has the potential to complement TNM staging and allow for specific tailoring of treatment interventions to improve clinical outcomes.

### Materials and Methods

### Cell Culture

Human non-small cell lung cancer (NSCLC) cell lines (adeno-and-squamous cell carcinoma) H358, SKMES1, H23, and H1975 were purchased from the ATCC. Cell line authentication was carried out using short tandem repeat profiling. NSCLC were maintained in DMEM or RPMI supplemented with 10% foetal bovine serum, 100 U/mL penicillin and 100 mg/mL streptomycin and incubated at 37° C. in 5% CO₂. Isogenic normal human bronchial epithelial cells (HBECs) were a gift from Dr. Jill Larsen^{19,23}. HBECs were cultured in keratinocyte serum free medium (KSFM), supplemented with EGF (5 μg/L) and bovine pituitary extract (50 mg/L), 37° C. in 5% CO₂. Cell conditioned media (CCM) from NSCLC cell lines were collected from cells cultured under normoxic (21% O₂) or hypoxic (2% O₂) conditions in serum-free media. CCM was collected from HBEC cells conditioned under normoxic or hypoxic conditions in KSFM depleted of bovine exosomes through overnight centrifugation at 100,000 g_{avg}.

### Antibodies and Reagents

The following antibodies were used for Western blotting: Calnexin (Cell Signaling Technology, 2679S), CD9 (Abcam, ab92726), CD63 (Abcam, ab8219), Flotillin-1 (BD Transduction Laboratories, 610821), HSP70 (Transduction Laboratories, 610608), TSG101 (Santa Cruz, sc-6037), VCP (Abcam, ab11433). Horseradish peroxidase (HRP)-conjugated secondary antibodies were purchased from Thermo Scientific. MAC2BP, PSMA2, and THBS1 ELISA DuoSets were purchased from R & D Systems, TNC ELISA kits were purchased from Abcam. qEV columns were purchased from Izon and stored in PBS (0.1% sodium azide) at 4° C. OptiPrep was purchased from Sigma-Aldrich. qPCR was carried out as previously described²⁴.

### Patients

The independent confirmation cohort included 20 patients who provided informed consent to participate in an ERB approved prospective trial of sequential FDG PET/CT prior to, during and after curative intent chemo-RT. As previously reported, eligibility for this trial included a staging ¹⁸F-FDG PET/CT, histological or cytological confirmation of stage I-III NSCLC, with an Eastern Cooperative Oncology Group (ECOG) performance status of 0-1²⁵. Exclusion criteria included previous thoracic radiotherapy and complete surgical tumour excision. Patients received concurrent chemo-RT in accordance with two standardised protocols. RT consisted of 60 Gy in 30 fractions over six weeks. One of two chemotherapy regimens was administered: either weekly carboplatin [area under curve, 2 intravenously] and paclitaxel [45 mg/m2 intravenously] for older patients or those with significant comorbidities; or cisplatin [50 mg/m2 intravenously] on days 1, 8, 29, and 36 and etoposide [50 mg/m2 intravenously] during weeks 1 and 5 for younger fitter patients. ¹⁸F-FDG PET/CT scans were acquired at baseline, Day 10, Day 24 and Day 90. Ongoing monitoring was performed with standard CT imaging at three monthly intervals for 12 months and six-monthly intervals thereafter.

### Exosome Isolation and Analysis

Exosomes were isolated and analysed as previously described^{7,17,26}. For exosome isolations from in vitro cell culture, CCM was centrifuged at 300 g for 10 minutes at 4° C. and filtered through 0.22 μm filters to remove floating cells and large extracellular vesicles. Clarified CCM was then concentrated to 500 μL and overlaid on a discontinuous iodixanol density gradient and centrifuged for 16 hours at 100,000 g_{avg} at 4° C. Exosome containing fractions were diluted to 20 mL in PBS and centrifuged at 100,000 g_{avg} at 4° C. for 2 hours. The resulting pellet was resuspended in PBS and stored at −80° C. until use. For the isolation of exosomes from human plasma, 3 mL of plasma was thawed at room temperature and prepared by removing remaining platelets and large vesicles by centrifugation at 1,500 g and 10,000 g, for 10 and 20 minutes respectively. Prepared plasma was subsequently diluted to 20 mL in PBS containing 2 mM EDTA and centrifuged at 100,000 g_{avg} at 4° C. for 2 hours. The resulting pellet was resuspended in 500 μL of PBS and overlaid on a size exclusion column followed by elution with PBS. Exosome containing fractions were collected and concentrated to 100 μL using Amicon® Ultra-4 10 kDA nominal molecular weight centrifugal filter units. Concentrated exosomes were stored at −80° C. until use. Exosome isolations from cell culture and human plasma were confirmed with western blot, tunable resistive pulse sensing (TRPS), and transmission electron microscopy as previously described^{7,17,26}.

Western Blot Analysis

Western blots were performed as previously described^{7,24}. Briefly, proteins were resolved by SDS-PAGE, transferred to polyvinylidene fluoride membranes, blocked in 5% non-fat powdered milk in PBS-T (0.5% Tween-20) and probed with antibodies. Protein bands were detected with enhanced chemiluminescence reagent (Amersham ECL Select). Protein bands were quantified with ImageJ and normalized to a loading control. To control for variability between gels, patient VCP levels were calibrated to 5 μg of hypoxic-derived SKMES1 exosomes from the same gel before being normalized to Flotillin-1 as a loading control.

### Immunohistochemistry

IHC analysis was carried out on formalin-fixed paraffin-embedded (FFPE) samples using automated staining and optimized methods. To assess expression for E-cadherin within tumour cells, the immunostained tumour cells were scored in regard to their staining intensity; 0 (negative), 1+ (weak), 2+ (moderate) and 3+ (strong).

### Mass Spectrometry

Exosome preparations were reduced by addition of 10 mM dithiothreitol (4° C. 1-hour, 22° C. 2 hours) in the presence of 2% SDS, protease inhibitors (SigmaAldrich, P8340) and 50 mM Tris.HCl pH 8.8. Samples were then alkylated by the addition of iodoacetamide to 25 mM (22° C. 1-hour) and methanol co-precipitated overnight at −20° C. with trypsin (1:100 enzyme:substrate). Pellets were resuspended in 10% acetonitrile, 40 mM ammonium bicarbonate and digested at 37° C. for 8 hours with further trypsin added after 2 hours (1:100 enzyme:substrate).

LCMS analysis of acidified digests (trifluoroacetic acid) was performed by interfacing a NanoAcquity UPLC (Waters) in front of an Elite Orbitrap ETD mass spectrometer (Thermo Fisher Scientific). Two micro-grams of digest was loaded onto a 20 mm×180 μm Symmetry C18 trap (Waters) and separated over 120 minutes on a 200 mm×75 μm, BEH130 1.7 μm column (Waters) using a series of linear gradients (buffer A: aqueous 0.1% formic acid; buffer B: 0.1% formic acid in acetonitrile) 2% B to 5% B over 5 minutes, 30% B over 75 minutes, 50% B over 10 minutes 95% B over 5 minutes and hold for 6 minutes, re-equilibrate in 2% B. Eluate from the column was introduced into the mass spectrometer through a 10 μm P200P coated silica emitter (New Objective) and Nanospray-Flex source (Proxeon Biosystems A/S). Source voltage 1.8 kV, heated capillary temperature 275° C., using a top 15 method MS acquired in the orbitrap at 120 000 resolution AGC 1E6, MS2 in the ion-trap AGC 1E4, 50 ms maximum injection time. MS1 lock mass of 445.120024 was used.

Protein identification and label-free quantification were performed using MaxQuant (version 1.4.1.2²⁷. MaxQuant was used to extract peak lists from the Xcalibur raw files (Thermo Fisher Scientific, Germany) and the embedded database search engine Andromeda²⁸ was used to assign peptide-to-spectrum matches (PSMs). The database searched consisted of the complete proteome for Homo sapiens (88,378 canonical sequences downloaded from www.uniprot.org August 2013). Reversed sequences and the MaxQuant contaminant database were also searched. Label-free quantification was performed, the instrument type was set to Orbitrap, the precursor mass tolerance was set to 20 ppm for the first search, 4.5 ppm for the main search, the fragment ion mass tolerance was set to 0.5 Da, the enzyme specificity was set to trypsin/P, a maximum of two missed cleavages were allowed, carbamidomethyl cysteine was specified as a fixed modification and acetylation of the protein N-terminal, deamidation of asparagine/glutamine and oxidation of methionine were specified as variable modifications. The second peptide search and match between runs were enabled with default settings. For identification, the PSM and protein level FDRs were set to 0.01. Default settings were applied for all other parameters. Protein inference and label-free quantification by spectral counting (including normalisation) were performed as previously described²⁹.

### Gene Set Enrichment Analysis

Gene set enrichment analysis (GSEA)³⁰, version 2.2.3, was used to identify enriched pathways in exosomes isolated from hypoxic SKMES1 cells as previously described¹⁵. Non-log 2 transformed protein intensity values of all proteins in exosomes derived from normoxic or hypoxic SKMES1 exosomes were analysed using the Molecular Signatures Database (MSigDB). Analysis was performed using the Hallmark gene sets database (version 5.2), Signal2Noise ranking metric, 1000 gene set permutations, and a weighted enrichment statistic. Results were considered significant with a false discovery rate (FDR)<0.05.

### Statistical Analysis

GraphPad Prism version 6.0, EdgeR version 2.6.10³¹, MedCalc version 16.8.4, and SPSS statistics were used for all calculations. Unpaired Student's t-test was used to calculate the difference in expression values of proteins from exosomes in vitro. The Mann Whitney test was used in patient-derived exosomes. A negative-binomial exact test was used to assess the mass spectrometry derived spectral counts, where the Benjamini-Hochberg adjustment was applied to control the FDR. Receiver operator characteristic (ROC) curves were used to determine the sensitivity and specificity of prognostic values. Threshold values were selected using Youden's index. Univariate analysis using the log-rank test was used to assess disease-free survival (Kaplan-Meier curves). Differences with p-values less than 0.05 were considered significant (*p<0.05, **p<0.01, ***p<0.001), with the exception of a FDR threshold of 0.001 and 0.05 for the spectral count and GSEA data respectively.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference.

TABLE 2
List of proteins upregulated in both H358 and SKMES1
hypoxic exosomes Commonly upregulated proteins
in NSCLC hypoxic exosomes (FDR < 0.01%).

| | Gene | H358 | SKMES1 |
|---|---|---|---|
| Entry | names | log2(N/H) | log2(N/H) |
| P53396 | ACLY | −0.930 | −0.984 |
| O00468 | AGRN | −0.996 | −1.100 |
| P05067 | APP | −1.661 | −1.741 |
| Q07954 | APR | −2.057 | −2.143 |
| P21333 | FLN1 | −0.621 | −0.756 |
| Q14697 | GANAB | −1.193 | −0.754 |
| P00367 | GLUD | −3.263 | −0.987 |
| P98160 | HSPG2 | −0.772 | −1.001 |
| Q16787 | LAMA3 | −1.287 | −2.351 |
| O15230 | LAMA5 | −1.230 | −0.950 |
| P07942 | LAMB1 | −0.908 | −0.859 |
| P11047 | LAMB2 | −0.932 | −1.068 |
| P55268 | LAMC1 | −1.091 | −0.848 |
| Q08380 | MAC2BP | −0.872 | −1.996 |
| Q9Y4K0 | LOXL2 | −2.027 | −1.391 |
| Q9NS15 | LTBP3 | −1.692 | −2.097 |
| P25786 | PSMA1 | −0.813 | −0.660 |
| P25787 | PSMA2 | −1.252 | −1.092 |
| P25788 | PSMA3 | −1.410 | −1.176 |
| P25789 | PSMA4 | −1.068 | −0.678 |
| P28066 | PSMA5 | −1.119 | −0.979 |
| P60900 | PSMA6 | −1.605 | −0.649 |
| P49721 | PSMB2 | −1.230 | −0.675 |
| P49720 | PSMB3 | −1.660 | −1.281 |
| P28070 | PSMB4 | −0.956 | −0.953 |
| Q99436 | PSMB7 | −1.401 | −0.860 |
| P28062 | PSMB8 | −1.276 | −1.050 |
| Q13813 | SPTAN1 | −1.288 | −0.675 |
| Q01082 | SPTBN1 | −1.420 | −0.794 |
| P07996 | THBS1 | −0.955 | −1.732 |
| P24821 | TNC | −0.805 | −0.863 |
| P55072 | VCP | −1.208 | −1.116 |

TABLE 3
Subcellular localisation of commonly upregulated
proteins in NSCLC hypoxic exosomes (FDR < 0.01%).

| Cytoplasm | Extracellular Space | Plasma Membrane |
|---|---|---|
| ACLY | HSPG2 | AGRN |
| FLN1 | LAMA3 | APP |
| GANAB | LAMA5 | APR |
| GLUD | LAMB1 | MAC2BP |
| PSMA1 | LAMC1 | NEAS |
| PSMA2 | LAMS | SPTB2 |
| PSMA3 | LOXL2 | |
| PSMA4 | LTBP3 | |
| PSMA5 | TNC | |
| PSMA6 | THBS1 | |
| PSMB2 | | |
| PSMB3 | | |
| PSMB4 | | |
| PSMB7 | | |
| PSMB8 | | |
| VCP | | |

TABLE 4
Patient information of discovery cohort.

| | | | Tumour | Time to | |
|---|---|---|---|---|---|
| | | | stage | recurrence | Follow-up |
| Age | Sex | Histology | (7th ed.) | (months) | (years) |
| 74 | Male | AdenoCA | Stage IB | 5.9 | 0 |
| 71 | Male | AdenoCA | Stage IIA | 7.866666667 | 0 |
| 50 | Male | AdenoCA | Stage IIA | 5.6 | 13.51780822 |
| 69 | Male | AdenoCA | Stage IIA | 8.066666667 | 0 |
| 54 | Male | AdenoCA | Stage IIIA | 12.13333333 | 0 |
| 72 | Female | AdenoCA | Stage IB | 14 | 0 |
| 68 | Male | AdenoCA | Stage IIB | 6.966666667 | 0 |
| 69 | Female | AdenoCA | Stage IIA | 9.233333333 | 0 |
| 62 | Male | AdenoCA | Stage IIIA | 6.433333333 | 0 |
| 66 | Male | AdenoCA | Stage IA | 12.1 | 0 |
| 66 | Female | AdenoCA | Stage IIA | 14.1 | 0 |
| 75 | Male | AdenoCA | Stage IIIA | 16.33333333 | 2.410958904 |
| 66 | Male | AdenoCA | Stage IA | N/A | 6.780821918 |
| 62 | Female | AdenoCA | Stage IIIA | N/A | 6.482191781 |
| 39 | Female | AdenoCA | Stage IB | N/A | 6.857534247 |
| 71 | Male | AdenoCA | Stage IIA | N/A | 4.471232877 |
| 75 | Male | AdenoCA | Stage IIIA | N/A | 4.430136986 |
| 64 | Male | AdenoCA | Stage IB | N/A | 7.402739726 |
| 65 | Male | AdenoCA | Stage IB | N/A | 6.739726027 |
| 59 | Female | AdenoCA | Stage IIA | N/A | 3.079452055 |
| 65 | Male | AdenoCA | Stage IA | N/A | 5.438356164 |
| 72 | Female | AdenoCA | Stage IA | N/A | 6.687671233 |
| 71 | Male | AdenoCA | Stage IA | N/A | 5.317808219 |
| 51 | Female | AdenoCA | Stage IIIA | N/A | 6.95890411 |
| 51 | Male | AdenoCA | Stage IIA | N/A | 4.695890411 |
| 57 | Female | AdenoCA | Stage IA | N/A | 5.679452055 |
| 54 | Male | AdenoCA | Stage IIB | N/A | 4.539726027 |
| 62 | Male | SCC | Stage IIIA | N/A | 3.57260274 |
| 67 | Male | AdenoCA | Stage IIA | N/A | 3.542465753 |
| 71 | Male | SCC | Stage IIB | N/A | 3.561643836 |
| 80 | Female | AdenoCA | Stage IA | N/A | 4.449315068 |
| 72 | Female | AdenoCA | Stage IA | N/A | 4.969863014 |

TABLE 5
Patient information of confirmation cohort.

| | | | | Tumour | | Time to |
|---|---|---|---|---|---|---|
| | | | | stage | Chemotherapy | recurrence |
| Patient ID | Age | Sex | Histology | (7th ed.) | regimen | (months) |
| 41 | 82 | Male | SCC | Stage IIIB | C/P | 4.633333 |
| 44 | 80 | Male | SCC | Stage IIIB | C/P | 41.13333 |
| 45 | 66 | Male | AdenoCA | Stage IIIA | C/E | 7.466667 |
| 46 | 67 | Female | N/A | Stage IB | C/P | 8.7 |
| 47 | 62 | Male | SCC | Stage IB | C/P | 24.8 |
| 48 | 72 | Male | Neuroendocrine | Stage IIIA | C/P | 4.633333 |
| 50 | 68 | Female | AdenoCA | Stage IIIA | C/P | 14.43333 |
| 51 | 70 | Male | AdenoCA | Stage IIIA | C/P | 6.866667 |
| 53 | 68 | Male | AdenoCA | Stage IIIB | C/E | 5.833333 |
| 54 | 77 | Male | AdenoCA | Stage IIIA | C/P | 4.5 |
| 55 | 31 | Female | AdenoCA | Stage IV | C/E | 18.23333 |
| 56 | 69 | Male | AdenoCA | Stage IIIB | C/E | 8.266667 |
| 57 | 74 | Male | AdenoCA | Stage IIB | C/P | 6.966667 |
| 58 | 68 | Male | AdenoCA | Stage IIIA | C/E | 32.96667 |
| 59 | 84 | Male | AdenoCA | Stage IIA | C/P | 31.06667 |
| 60 | 64 | Male | AdenoCA | Stage IIIB | C/E | 8.9 |
| 61 | 80 | Male | SCC | Stage IIIA | C/P | 30.33333 |
| 62 | 65 | Male | SCC | Stage IIIA | C/P | 4.733333 |
| 63 | 64 | Male | SCC | Stage IIIB | C/E | 0.466667 |
| 65 | 68 | Female | SCC | Stage IIB | C/E | 26.66667 |

### REFERENCES

1. Torre, L. A., Bray, F., Siegel, R. L., Ferlay, J., Lortet-Tieulent, J. & Jemal, A. Global cancer statistics, 2012. CA: a cancer journal for clinicians 65, 87-108 (2015).
2. Molina, J. R., Yang, P., Cassivi, S. D., Schild, S. E. & Adjei, A. A. Non-small cell lung cancer: epidemiology, risk factors, treatment, and survivorship. Mayo Clinic proceedings 83, 584-594 (2008).
3. Melo, S. A., Luecke, L. B., Kahlert, C., Fernandez, A. F., Gammon, S. T., Kaye, J., et al. Glypican-1 identifies cancer exosomes and detects early pancreatic cancer. Nature 523, 177-182 (2015).
4. Lobb, R. J., Lima, L. G. & Moller, A. Exosomes: Key mediators of metastasis and pre-metastatic niche formation. Seminars in cell &developmental biology (2017).
5. Vaupel, P. & Mayer, A. Hypoxia in cancer: significance and impact on clinical outcome. Cancer metastasis reviews 26, 225-239 (2007).
6. Hockel, M. & Vaupel, P. Tumor hypoxia: definitions and current clinical, biologic, and molecular aspects. Journal of the National Cancer Institute 93, 266-276 (2001).
7. Lobb, R. J., Becker, M., Wen, S. W., Wong, C. S., Wiegmans, A. P., Leimgruber, A., et al. Optimized exosome isolation protocol for cell culture supernatant and human plasma. J Extracell Vesicles 4, 27031 (2015).
8. Lobb, R. & Moller, A. Size Exclusion Chromatography: A Simple and Reliable Method for Exosome Purification. Methods in molecular biology 1660, 105-110 (2017).
9. Valle, C. W., Min, T., Bodas, M., Mazur, S., Begum, S., Tang, D., et al. Critical role of VCP/p97 in the pathogenesis and progression of non-small cell lung carcinoma. PloS one 6, e29073 (2011).
10. Denlinger, C. E., Rundall, B. K., Keller, M. D. & Jones, D. R. Proteasome inhibition sensitizes non-small-cell lung cancer to gemcitabine-induced apoptosis. The Annals of thoracic surgery 78, 1207-1214; discussion 1207-1214 (2004).
11. Tang, Y. A., Chen, C. H., Sun, H. S., Cheng, C. P., Tseng, V. S., Hsu, H. S., et al. Global Oct4 target gene analysis reveals novel downstream PTEN and TNC genes required for drug-resistance and metastasis in lung cancer. Nucleic acids research 43, 1593-1608 (2015).
12. Oskarsson, T., Acharyya, S., Zhang, X. H., Vanharanta, S., Tavazoie, S. F., Morris, P. G., et al. Breast cancer cells produce tenascin C as a metastatic niche component to colonize the lungs. Nature medicine 17, 867-874 (2011).
13. Kudo-Saito, C., Shirako, H., Takeuchi, T. & Kawakami, Y. Cancer metastasis is accelerated through immunosuppression during Snail-induced EMT of cancer cells. Cancer cell 15, 195-206 (2009).
14. Marchetti, A., Tinari, N., Buttitta, F., Chella, A., Angeletti, C. A., Sacco, R., et al. Expression of 90K (Mac-2 BP) correlates with distant metastasis and predicts survival in stage I non-small cell lung cancer patients. Cancer Res 62, 2535-2539 (2002).
15. Lobb, R. J., Hastie, M. L., Norris, E. L., van Amerongen, R., Gorman, J. J. & Moller, A. Oncogenic transformation of lung cells results in distinct exosome protein profile similar to the cell of origin. Proteomics (2017).
16. Thiery, J. P., Acloque, H., Huang, R. Y. & Nieto, M. A. Epithelial-mesenchymal transitions in development and disease. Cell 139, 871-890 (2009).
17. Lobb, R. J., van Amerongen, R., Wiegmans, A., Ham, S., Larsen, J. E. & Moller, A. Exosomes derived from mesenchymal non-small cell lung cancer cells promote chemoresistance. International journal of cancer (2017).
18. Fischer, K. R., Durrans, A., Lee, S., Sheng, J., Li, F., Wong, S. T., et al. Epithelial-to-mesenchymal transition is not required for lung metastasis but contributes to chemoresistance. Nature 527, 472-476 (2015).
19. Larsen, J. E., Nathan, V., Osborne, J. K., Farrow, R. K., Deb, D., Sullivan, J. P., et al. ZEB1 drives epithelial-to-mesenchymal transition in lung cancer. The Journal of clinical investigation 126, 3219-3235 (2016).
20. Zheng, X., Carstens, J. L., Kim, J., Scheible, M., Kaye, J., Sugimoto, H., et al. Epithelial-to-mesenchymal transition is dispensable for metastasis but induces chemoresistance in pancreatic cancer. Nature 527, 525-530 (2015).
21. Sceneay, J., Chow, M. T., Chen, A., Halse, H. M., Wong, C. S. F., Andrews, D. M., et al. Primary Tumor Hypoxia Recruits CD11b+/Ly6Cmed/Ly6G+ Immune Suppressor Cells and Compromises NK Cell Cytotoxicity in the Premetastatic Niche. Cancer Research 72, 3906 (2012).
22. Kalluri, R. & Weinberg, R. A. The basics of epithelial-mesenchymal transition. The Journal of clinical investigation 119, 1420-1428 (2009).
23. Sato, M., Vaughan, M. B., Girard, L., Peyton, M., Lee, W., Shames, D. S., et al. Multiple oncogenic changes (K-RAS(V12), p53 knockdown, mutant EGFRs, p16 bypass, telomerase) are not sufficient to confer a full malignant phenotype on human bronchial epithelial cells. Cancer Res 66, 2116-2128 (2006).
24. Lobb, R. J., van Amerongen, R., Wiegmans, A., Ham, S., Larsen, J. E. & Moller, A. Exosomes derived from mesenchymal non-small cell lung cancer cells promote chemoresistance. International journal of cancer 141, 614-620 (2017).
25. Everitt, S. J., Ball, D. L., Hicks, R. J., Callahan, J., Plumridge, N., Collins, M., et al. Differential (18)F-FDG and (18)F-FLT Uptake on Serial PET/CT Imaging Before and During Definitive Chemoradiation for Non-Small Cell Lung Cancer. Journal of nuclear medicine: official publication, Society of Nuclear Medicine 55, 1069-1074 (2014).
26. Wen, S. W., Sceneay, J., Lima, L. G., Wong, C. S., Becker, M., Krumeich, S., et al. The biodistribution and immune suppressive effects of breast cancer-derived exosomes. Cancer Research, canres. 0868.2016 (2016).
27. Cox, J. & Mann, M. MaxQuant enables high peptide identification rates, individualized p.p.b.-range mass accuracies and proteome-wide protein quantification. Nature biotechnology 26, 1367-1372 (2008).
28. Cox, J., Neuhauser, N., Michalski, A., Scheltema, R. A., Olsen, J. V. & Mann, M. Andromeda: a peptide search engine integrated into the MaxQuant environment. Journal of proteome research 10, 1794-1805 (2011).
29. Dave, K. A., Norris, E. L., Bukreyev, A. A., Headlam, M. J., Buchholz, U. J., Singh, T., et al. A comprehensive proteomic view of responses of A549 type II alveolar epithelial cells to human respiratory syncytial virus infection. Molecular & cellular proteomics: MCP 13, 3250-3269 (2014).
30. Subramanian, A., Tamayo, P., Mootha, V. K., Mukherjee, S., Ebert, B. L., Gillette, M. A., et al. Gene set enrichment analysis: a knowledge-based approach for interpreting genome-wide expression profiles. Proceedings of the National Academy of Sciences of the United States of America 102, 15545-15550 (2005).
31. Robinson, M. D., McCarthy, D. J. & Smyth, G. K. edgeR: a Bioconductor package for differential expression analysis of digital gene expression data. Bioinformatics 26, 139-140 (2010).

1. A method of determining the aggressiveness of a cancer in a subject, said method including the step of determining an expression level of one or a plurality of markers in an exosome sample of the subject, wherein the markers comprise one or more proteins selected from the group consisting of Galectin-3-Binding Protein, Proteasome subunit alpha type-2, Neutral alpha-glucosidase AB and any combination thereof and an expression level of the one or plurality of markers indicates or correlates with a level of aggressiveness of the cancer.

2. A method of determining a prognosis for a cancer in a subject, said method including the step of determining an expression level of one or a plurality of markers in an exosome sample of the subject, wherein the markers comprise one or more proteins selected from the group consisting of Galectin-3-Binding Protein, Proteasome subunit alpha type-2, Neutral alpha-glucosidase AB and any combination thereof and an expression level of the one or plurality of markers indicates or correlates with a less or more favourable prognosis for said cancer.

3. The method of claim 1, wherein a relatively decreased expression level of the one or plurality of markers indicates or correlates with a less aggressive cancer; and/or a relatively increased expression level of the one or plurality of markers indicates or correlates with a highly aggressive cancer.

4. The method of claim 1, which includes the further step of diagnosing said subject as having: (i) a highly aggressive cancer or a less aggressive cancer.

5-6. (canceled)

7. A method of predicting the responsiveness of a cancer to an anti-cancer treatment in a subject, said method including the step of determining an expression level of one or a plurality of markers in an exosome sample of the subject, wherein the markers comprise one or more proteins selected from the group consisting of Galectin-3-Binding Protein, Proteasome subunit alpha type-2, Neutral alpha-glucosidase AB and any combination thereof and an altered or modulated expression level of the one or plurality of markers indicates or correlates with relatively increased or decreased responsiveness of the cancer to the anti-cancer treatment.

8. The method of claim 7, which includes the further step of treating the cancer in the subject.

9. A method of treating cancer in a subject, said method including the step of determining an expression level of one or a plurality of markers in an exosomal sample of the subject, wherein the markers comprise one or more proteins selected from the group consisting of Galectin-3-Binding Protein, Proteasome subunit alpha type-2, Neutral alpha-glucosidase AB and any combination thereof and based on the determination made, initiating, continuing, modifying or discontinuing an anti-cancer treatment.

10. The method of claim 7, wherein the anti-cancer treatment comprises administration to the subject of a therapeutically effective amount of an anti-cancer agent that decreases the expression and/or an activity of the one or plurality of markers.

11-12. (canceled)

13. The method of claim 1, further including the step of obtaining the exosomal sample from the subject.

14-16. (canceled)

17. A method for identifying or producing an agent for use in the treatment of cancer in a subject including the steps of:
(a) contacting a cell that expresses a marker selected from the group consisting of Galectin-3-Binding Protein, Proteasome subunit alpha type-2, Neutral alpha-glucosidase AB and any combination thereof with a candidate agent; and
(b) determining whether the candidate agent modulates the expression and/or an activity of the marker.

18-19. (canceled)

20. The method of claim 1, further including the step of determining an expression level of one or a plurality of additional markers selected from the group consisting of Transitional endoplasmic reticulum ATPase, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof.

21-23. (canceled)

24. The method of claim 2, wherein a relatively decreased expression level of the one or plurality of markers indicates or correlates with a more favourable prognosis; and/or a relatively increased expression level of the one or plurality of markers indicates or correlates with a less favourable prognosis.

25. The method of claim 2, which includes the further step of diagnosing said subject as having a less favourable prognosis or a more favourable prognosis.

26. The method of claim 9, wherein the anti-cancer treatment comprises administration to the subject of a therapeutically effective amount of an anti-cancer agent that decreases the expression and/or an activity of the one or plurality of markers.

27. The method of claim 2, further including the step of obtaining the exosomal sample from the subject.

28. The method of claim 7, further including the step of obtaining the exosomal sample from the subject.

29. The method of claim 9, further including the step of obtaining the exosomal sample from the subject.

30. The method of claim 2, further including the step of determining an expression level of one or a plurality of additional markers selected from the group consisting of Transitional endoplasmic reticulum ATPase, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof.

31. The method of claim 7, further including the step of determining an expression level of one or a plurality of additional markers selected from the group consisting of Transitional endoplasmic reticulum ATPase, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof.

32. The method of claim 9, further including the step of determining an expression level of one or a plurality of additional markers selected from the group consisting of Transitional endoplasmic reticulum ATPase, 60 kDa heat shock protein, Lysyl oxidase homolog 2, Tenascin C, Fatty acid synthase, Agrin, Aspartyl aminopeptidase, Proteasome subunit alpha type-1, Proteasome subunit alpha type-3, Proteasome subunit alpha type-4, Proteasome subunit alpha type-5, Proteasome subunit alpha type-6, Proteasome subunit beta type-1, Proteasome subunit beta type-2, Proteasome subunit beta type-3, Proteasome subunit beta type-4, Proteasome subunit beta type-5, Proteasome subunit beta type-6, Proteasome subunit beta type-7, Proteasome subunit beta type-8, Thrombospondin-1, Latent Transforming Growth Factor Beta Binding Protein 3 and any combination thereof.
